(19) United States
(12) Patent Application Publication
(10) Pub. No.: US 2024/0254092 A1
(43) Pub. Date: Aug. 1, 2024

(54) BIFUNCTIONAL CHELATORS AND CONJUGATES

(71) Applicant: ACTINIUM PHARMACEUTICALS, INC., New York, NY (US)

(72) Inventors: Le-Cun XU, Yonkers, NY (US); Denis BECKFORD VERA, Valhalla, NY (US)

(73) Assignee: ACTINIUM PHARMACEUTICALS, INC., New York, NY (US)

(21) Appl. No.: 18/610,927

(22) Filed: Mar. 20, 2024

Related U.S. Application Data
(63) Continuation of application No. 18/482,912, filed on Oct. 8, 2023, now Pat. No. 11,964,948.
Application No. 18/482,912 is a continuation of application No. PCT/US2023/068062, filed on Jun. 7, 2023.
(60) Provisional application No. 63/487,789, filed on Mar. 1, 2023.
(60) Provisional application No. 63/408,970, filed on Sep. 22, 2022.
(60) Provisional application No. 63/349,833, filed on Jun. 7, 2022.

(51) Int. Cl. C07D 257/02; A61K 51/04; A61K 51/10; C07K 16/28

(52) U.S. Cl. CPC C07D 257/02; A61K 51/0482; A61K 51/1093; C07K 16/283; C07B 2200/05

(57) ABSTRACT

Provided are new bifunctional chelators for use in the manufacture of radiolabeled targeting agents for therapeutic or diagnostic use. Also provided are conjugates of the new bifunctional chelators with various cancer antigen targeting agents.

### CROSS-REFERENCE TO RELATED APPLICATIONS

This application is a continuation of U.S. patent application Ser. No. 18/482,912 filed on Oct. 8, 2023, entitled BIFUNCTIONAL CHELATORS AND CONJUGATES, which claims priority to and the benefit of International Application No. PCT/US2023/068062 filed on Jun. 7, 2023, entitled BIFUNCTIONAL CHELATORS AND CONJUGATES, which claims priority to and the benefit of U.S. Provisional Patent Application No. 63/349,833 filed on Jun. 7, 2022, entitled BIFUNCTIONAL CHELATORS, U.S. Provisional Patent Application No. 63/408,970 filed on Sep. 22, 2022, entitled BIFUNCTIONAL CHELATORS, and U.S. Provisional Patent Application No. 63/487,789 filed on Mar. 1, 2023, entitled BIFUNCTIONAL CHELATORS AND CONJUGATES, which are expressly incorporated herein by reference in their entireties.

### SEQUENCE LISTING

The present disclosure contains a Sequence Listing which has been submitted electronically in XML format and is hereby incorporated by reference in its entirety. Said XML copy, created on Jun. 7, 2023, is named PT23-045PCT_SL_ST26.xml and is 184,148 bytes in size.

### FIELD OF THE INVENTION

The present disclosure relates to the field of bifunctional chelators and linkers.

### BACKGROUND

Bifunctional chelators are compounds that include a metal (or metalloid) chelating moiety and a reactive group that can mediate conjugation to a target molecule such as a protein. Dodecane tetraacetic acid (DOTA), IUPAC name 2,2′,2″,2″-(1,4,7,10-Tetraazacyclododecane-1,4,7,10-tetrayl)tetraacetic acid, is a macrocyclic compound capable of chelating radiometals such as ⁹⁰Y, ²²⁵Ac, and ¹⁷⁷Lu. Bifunctional DOTA chelators known in the art include, for example, S-2-(4-Isothiocyanatobenzyl)-1,4,7,10-tetraazacyclododecane tetraacetic acid (also known as p-SCN-Bn-DOTA) and 1,4,7,10-Tetraazacyclododecane-1,4,7,10-tetraacetic acid mono-N-hydroxysuccinimide ester (also known as DOTA-NHS-ester).

### BRIEF SUMMARY

The present disclosure provides novel bifunctional chelator compounds of formula (A), or a metal complex thereof:

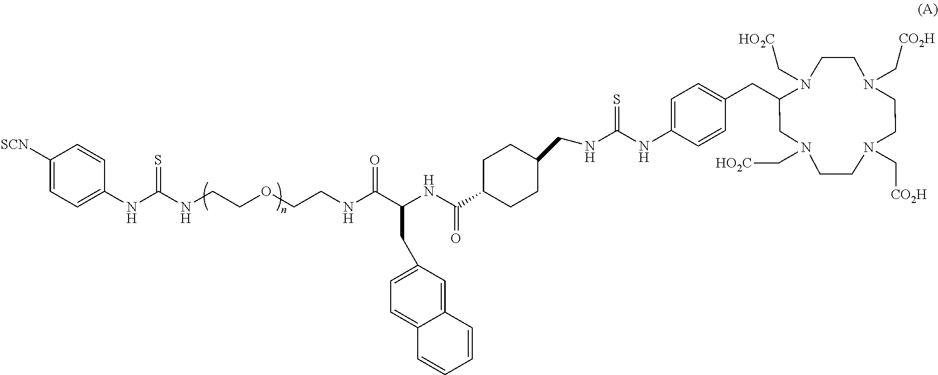

wherein n is 1 or an integer greater than 1, such as an integer from 1 to 1000 or any integer value therein or any subrange of integers therein, such as from 1 to 100 or 1 to 20. For example, n may be 1, 2, 3, 4, 5, 6, 7, 8, 9, or 10.

The present disclosure provides novel bifunctional chelator compounds of formula (B), or a metal complex thereof:

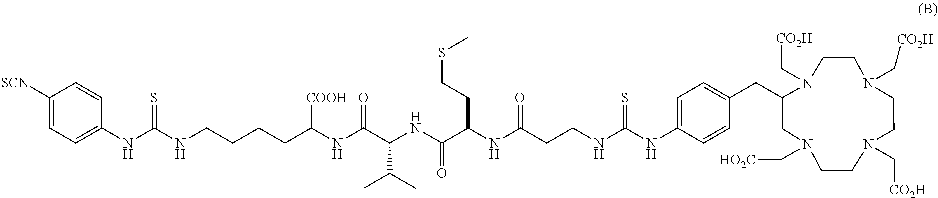

The present disclosure provides novel bifunctional chelator compounds having a formula including a metal chelator and at least one component selected from: a reactive group for conjugation of the bifunctional chelator to a target molecule such as a protein or a peptide, a benzyl group, at least one amino acid and/or amino acid derivative, a chelator moiety, a PEG spacer, a non-aromatic cyclic hydrocarbon, ethyleneamine, and thiourea, wherein when more than one component is included, the reactive group is a terminal component and any additional components may be in any order.

The present disclosure further provides novel bifunctional chelator compounds having a formula as shown in any one of FIGS. 3-17, i.e., compounds C-Q, or metal complexes thereof.

The present disclosure further provides a conjugated molecule formed by reacting one of the aforementioned bifunctional chelator compounds that includes a N-hydroxysuccinimide ester/NHS ester (abbreviated “NHS” herein) or thiocyanate (abbreviated “SCN” herein) reactive group and a molecule including one or more primary amine groups, including, but not limited to, a peptide, such as a synthetic peptide, or a protein, such as a recombinant protein.

The present disclosure provides a method for conjugating a chelator to a molecule including one or more primary amine groups, such as a protein or a peptide that includes the step of reacting the molecule with one of the aforementioned bifunctional chelator compounds that includes a NHS or SCN reactive group to form a chelator-conjugated molecule.

The present disclosure provides a conjugated molecule formed by reacting one of the aforementioned bifunctional chelator compounds that includes a phenyloxadiazolyl methylsulfone (abbreviated “PODS” herein) reactive group or derivative thereof and a molecule including one or more free thiol groups, including, but not limited to, a peptide, such as a synthetic peptide, or a protein, such as a recombinant protein.

Also provided by this disclosure is a method for conjugating a chelator to a molecule including one or more free thiol groups, such as a protein or a peptide, that includes the step of reacting the molecule with one of the aforementioned bifunctional chelator compounds that includes a PODS reactive group or derivative thereof to form a chelator-conjugated molecule. The method may include a prior or concurrent step of forming a free thiol group for reaction with a PODS reactive group by reducing a disulfide bond present in a molecule or connecting different molecules.

The molecules that are conjugated with the bifunctional chelator compounds may include antibodies such as monoclonal antibodies, or antibody chains, such as immunoglobulin heavy chains and/or immunoglobulin light chains, and/or the variable regions of such heavy or light chains. The molecules that are conjugated with the bifunctional chelator compounds may include antigen binding fragments of monoclonal antibodies such as Fab fragments or Fab₂ fragments, or corresponding scFv molecules.

Additional features, advantages, and aspects of the present disclosure may be set forth or apparent from consideration of the following detailed description, drawings if any, and claims. Moreover, it is to be understood that both the foregoing summary of the present disclosure and the following detailed description are exemplary and intended to provide further explanation without limiting the scope of the invention as claimed.

### BRIEF DESCRIPTION OF THE DRAWINGS

FIG. 1 shows a bifunctional chelator compound of Formula A.

FIG. 2 shows a bifunctional chelator compound of Formula B.

FIG. 3 shows a bifunctional chelator compound of Formula C.

FIG. 4 shows a bifunctional chelator compound of Formula D.

FIG. 5 shows a bifunctional chelator compound of Formula E.

FIG. 6 shows a bifunctional chelator compound of Formula F.

FIG. 7 shows a bifunctional chelator compound of Formula G.

FIG. 8 shows a bifunctional chelator compound of Formula H.

FIG. 9 shows a bifunctional chelator compound of Formula I.

FIG. 10 shows a bifunctional chelator compound of Formula J.

FIG. 11 shows a bifunctional chelator compound of Formula K.

FIG. 12 shows a bifunctional chelator compound of Formula L.

FIG. 13 shows a bifunctional chelator compound of Formula M.

FIG. 14 shows a bifunctional chelator compound of Formula N.

FIG. 15 shows a bifunctional chelator compound of Formula O.

FIG. 16 shows a bifunctional chelator compound of Formula P.

FIG. 17 shows a bifunctional chelator compound of Formula Q.

FIG. 18 shows a synthetic scheme of the present disclosure for the synthesis of the bifunctional chelator compound of Formula C shown in FIG. 3.

FIG. 19 shows a synthetic scheme of the present disclosure for the synthesis of a bifunctional chelator compound of Formula A shown in FIG. 1.

FIG. 20 shows a synthetic scheme of the present disclosure for the synthesis of a bifunctional chelator compound of Formula A shown in FIG. 1.

FIG. 21 shows a synthetic scheme of the present disclosure for the synthesis of a bifunctional chelator compound of Formula B shown in FIG. 2.

FIG. 22 shows conjugation of a bifunctional chelator molecule having a SCN reactive group to a primary amine of an amine presenting molecule.

FIG. 23 shows conjugation of a bifunctional chelator molecule having a PODS reactive group to a thiol of a thiol presenting molecule.

FIG. 24 shows conjugation of a bifunctional chelator molecule having a NHS reactive group to a primary amine of an amine presenting molecule.

### DETAILED DESCRIPTION

The present disclosure provides bifunctional chelator compounds. The bifunctional chelator compounds of the present disclosure may be used for the manufacture of radiolabeled targeting agents for therapeutic and/or diagnostic use. The present disclosure further provides methods of conjugation of the bifunctional chelator compounds to primary amines or thiols, such as amines or thiols of a peptide or protein, and compositions including the resulting conjugates, and methods for radiolabeling the resulting conjugates.

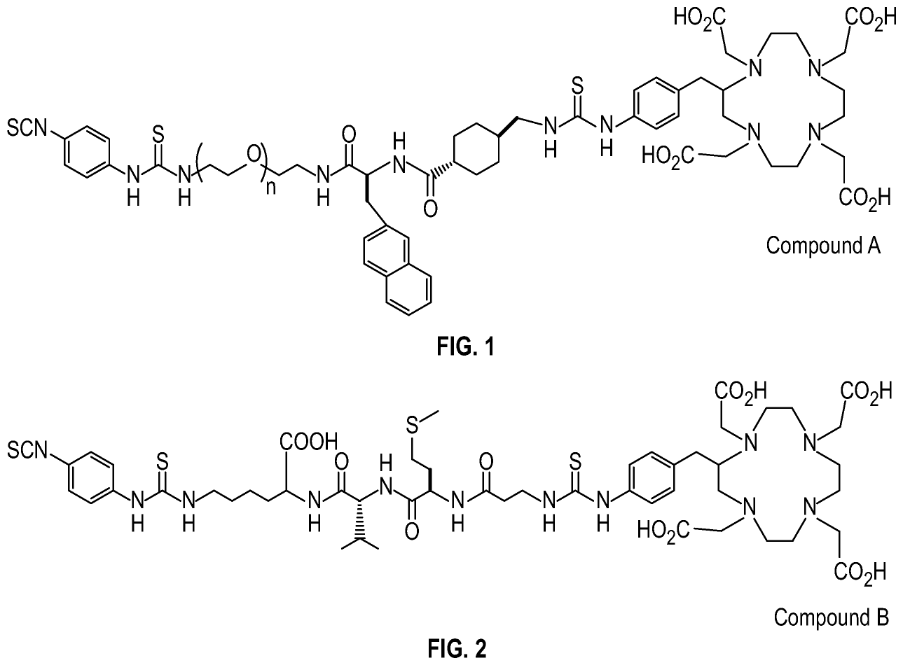

The bifunctional chelator compounds of the present disclosure may include a compound having the formula (A) (FIG. 1), or a metal complex thereof:

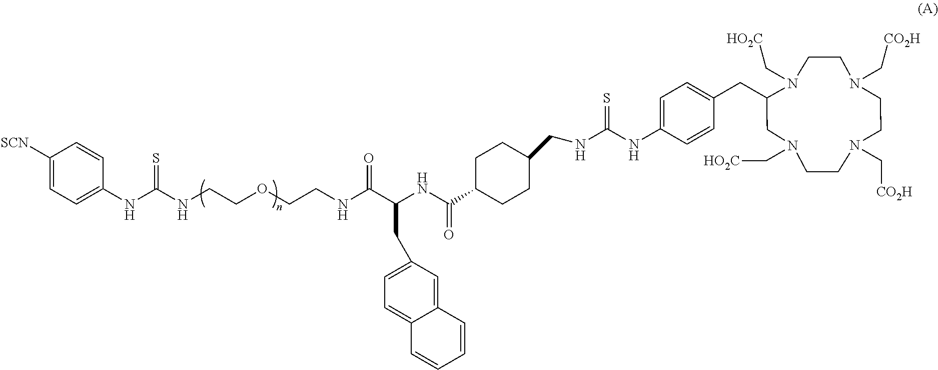

having a PEGₙ spacer, wherein n is 1 or an integer greater than 1, such as from 1 to 1000 or any integer value therein or any subrange of integers therein, such as from 1 to 100 or 1 to 20. The PEGₙ spacer may in addition or alternatively be defined by molecular weight or average molecular weight. For example, the average molecular weight of the PEG element may be 0.1 KD to 1 KD, 1 KD to 5 KD, 5 KD to 15 KD, 15 KD to 25 KD, 25 KD to 35 KD, or 35 KD to 50 KD.

The bifunctional chelator compounds of the present disclosure may include a compound having the formula (B) (FIG. 2), or a metal complex thereof:

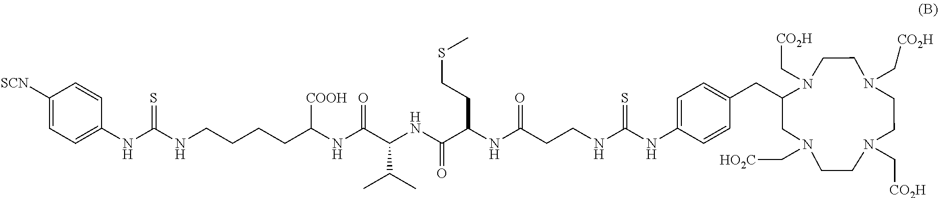

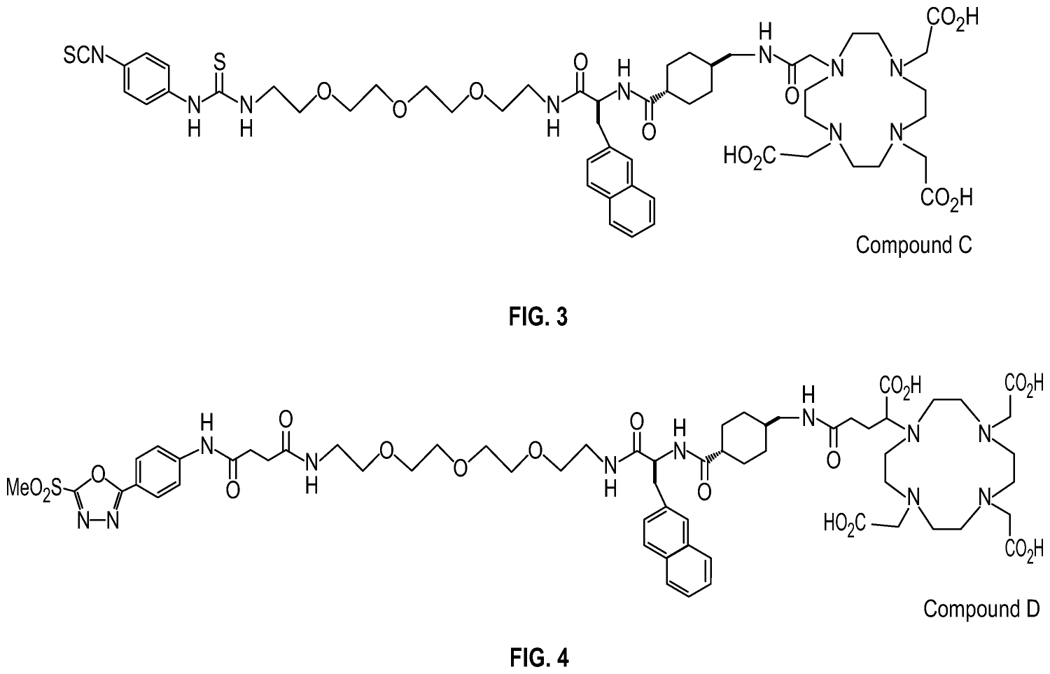

The bifunctional chelator compounds of the present disclosure may include a compound having the formula (C) (FIG. 3), or a metal complex thereof:

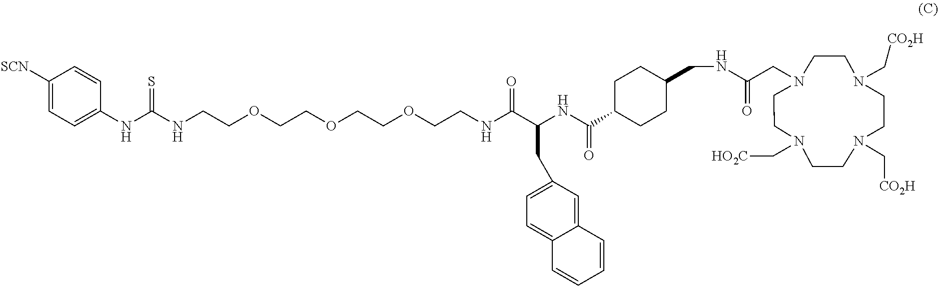

The bifunctional chelator compounds of the present disclosure may include a compound having the formula (D) (FIG. 4), or a metal complex thereof:

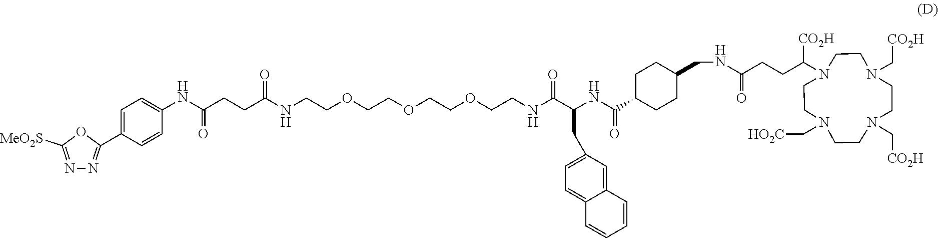 [SMILES: CS(=O)(=O)c1nnc(-c2ccc(NC(=O)CCC(=O)NCCOCCOCCOCCNC(=O)[C@H](Cc3ccc4ccccc4c3)NC(=O)[C@H]3CC[C@H](CNC(=O)CCC(C(=O)O)N4CCN(CC(=O)O)CCN(CC(=O)O)CCN(CC(=O)O)CC4)CC3)cc2)o1]

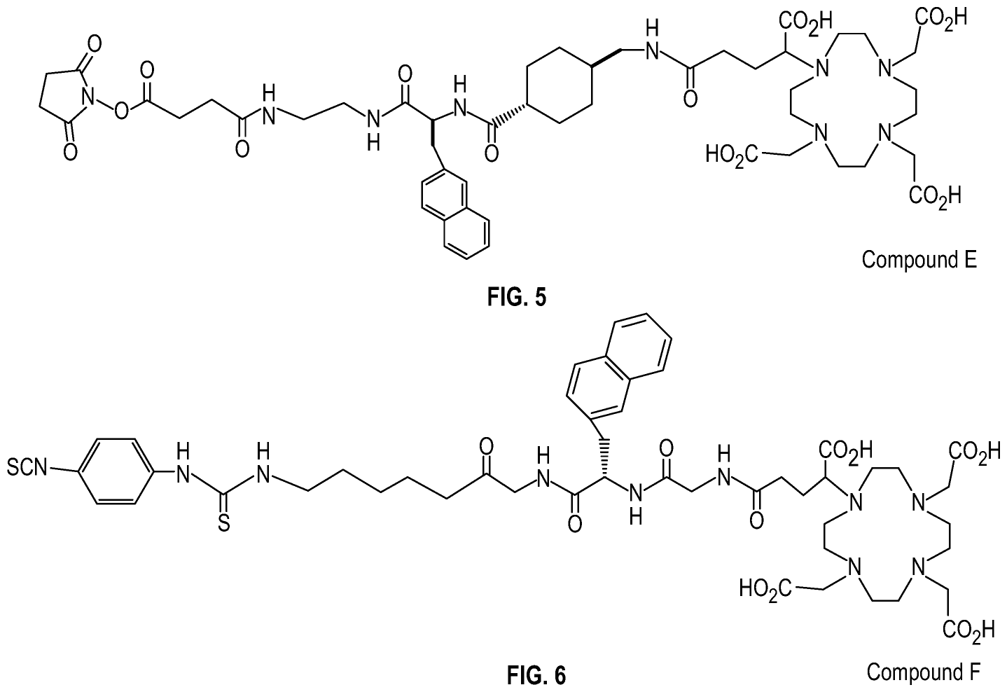

The bifunctional chelator compounds of the present disclosure may include a compound having the formula (E) (FIG. 5), or a metal complex thereof:

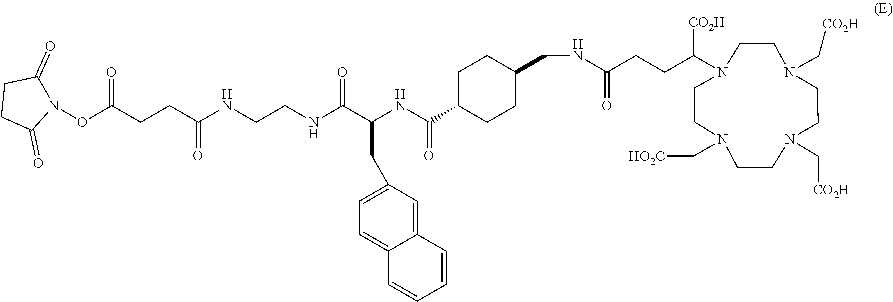 [SMILES: O=C(O)CN1CCN(CC(=O)O)CCN(C(CCC(=O)NC[C@H]2CC[C@H](C(=O)N[C@@H](Cc3ccc4ccccc4c3)C(=O)NCCNC(=O)CCC(=O)ON3C(=O)CCC3=O)CC2)C(=O)O)CCN(CC(=O)O)CC1]

The bifunctional chelator compounds of the present disclosure may include a compound having the formula (F) (FIG. 6), or a metal complex thereof:

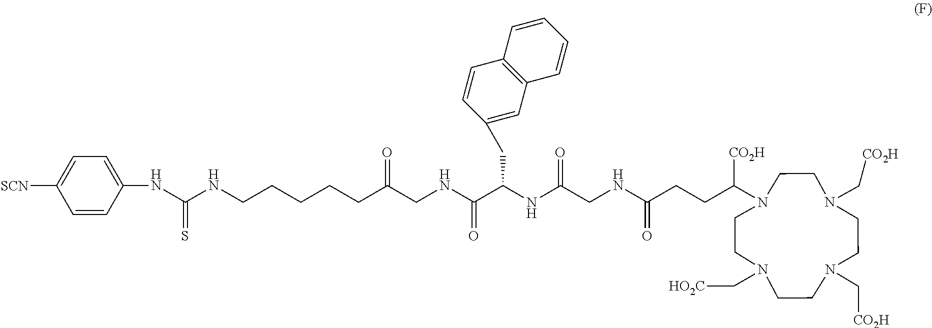

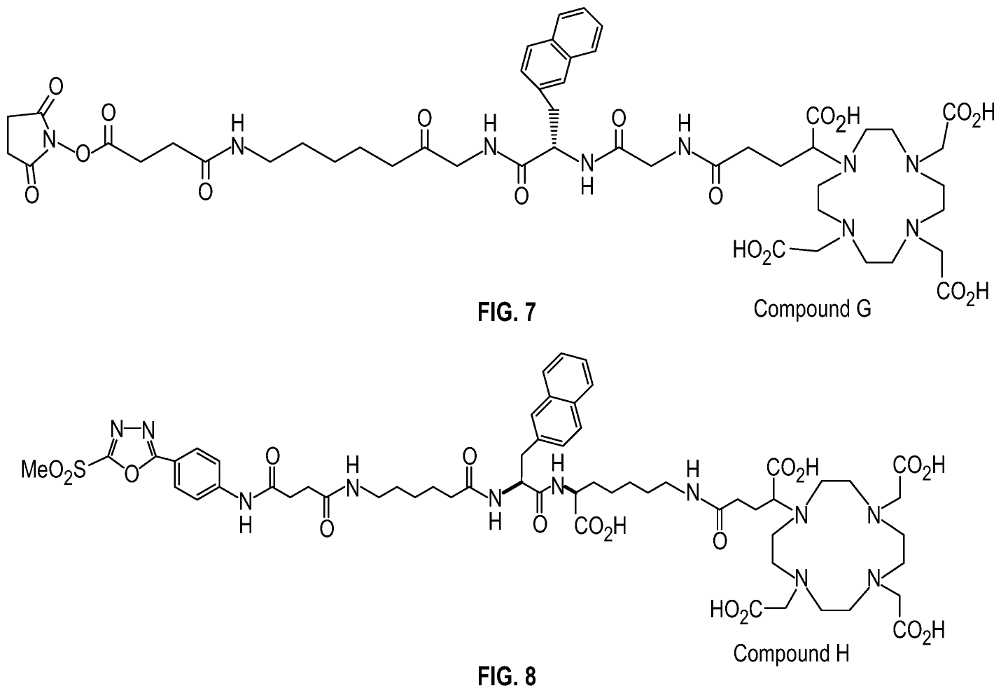

The bifunctional chelator compounds of the present disclosure may include a compound having the formula (G) (FIG. 7), or a metal complex thereof:

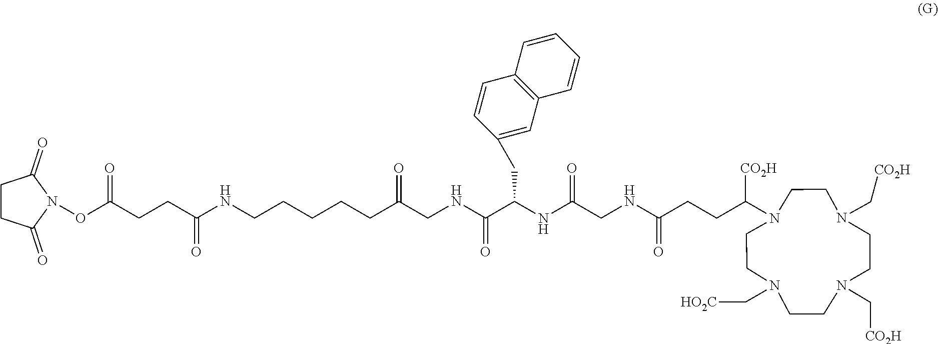 [SMILES: O=C(O)CN1CCN(CC(=O)O)CCN(C(CCC(=O)NCC(=O)N[C@@H](Cc2ccc3ccccc3c2)C(=O)NCC(=O)CCCCCNC(=O)CCC(=O)ON2C(=O)CCC2=O)C(=O)O)CCN(CC(=O)O)CC1]

The bifunctional chelator compounds of the present disclosure may include a compound having the formula (H) (FIG. 8), or a metal complex thereof:

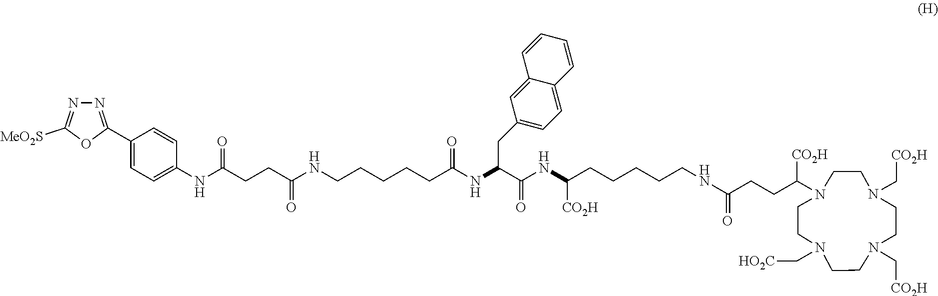 [SMILES: CS(=O)(=O)c1nnc(-c2ccc(NC(=O)CCC(=O)NCCCCCC(=O)N[C@@H](Cc3ccc4ccccc4c3)C(=O)N[C@@H](CCCCCNC(=O)CCC(C(=O)O)N3CCN(CC(=O)O)CCN(CC(=O)O)CCN(CC(=O)O)CC3)C(=O)O)cc2)o1]

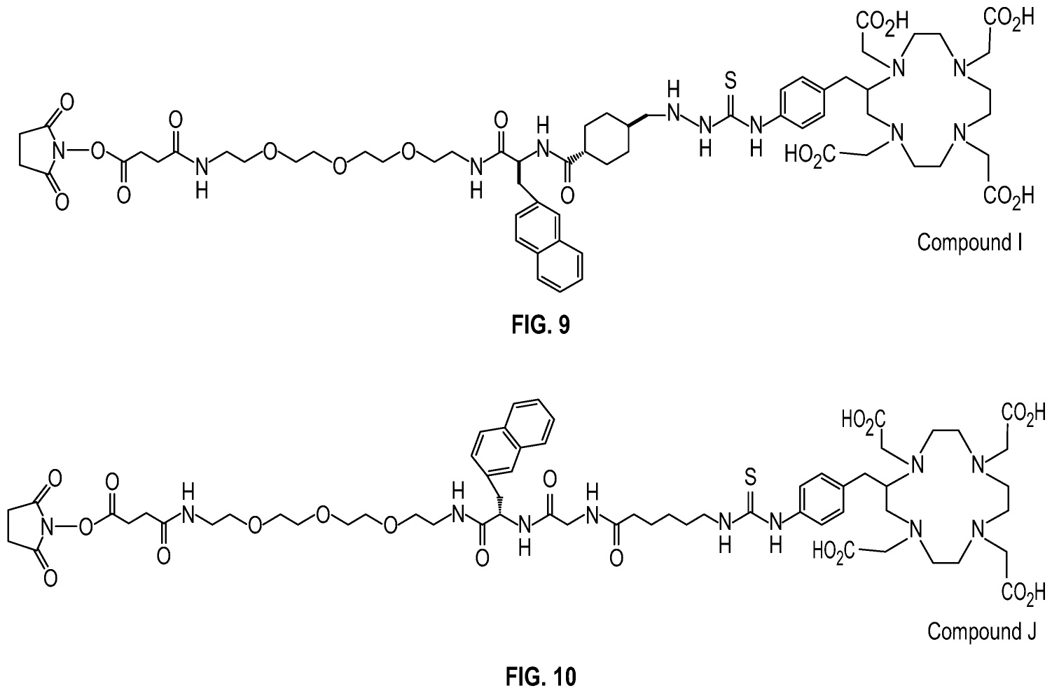

The bifunctional chelator compounds of the present disclosure may include a compound having the formula (I) (FIG. 9), or a metal complex thereof:

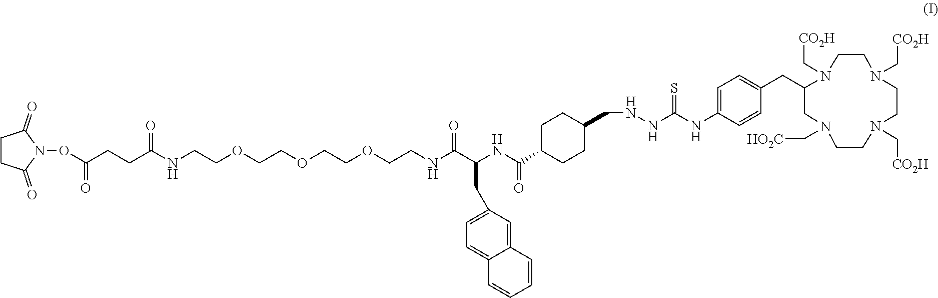 [SMILES: O=C(O)CN1CCN(CC(=O)O)CCN(CC(=O)O)C(Cc2ccc(NC(=S)NNC[C@H]3CC[C@H](C(=O)N[C@@H](Cc4ccc5ccccc5c4)C(=O)NCCOCCOCCOCCNC(=O)CCC(=O)ON4C(=O)CCC4=O)CC3)cc2)CN(CC(=O)O)CC1]

The bifunctional chelator compounds of the present disclosure may include a compound having the formula (J) (FIG. 10), or a metal complex thereof:

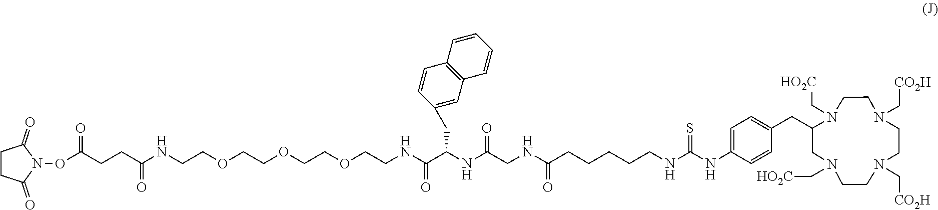 [SMILES: O=C(O)CN1CCN(CC(=O)O)CCN(CC(=O)O)C(Cc2ccc(NC(=S)NCCCCCC(=O)NCC(=O)N[C@@H](Cc3ccc4ccccc4c3)C(=O)NCCOCCOCCOCCNC(=O)CCC(=O)ON3C(=O)CCC3=O)cc2)CN(CC(=O)O)CC1]

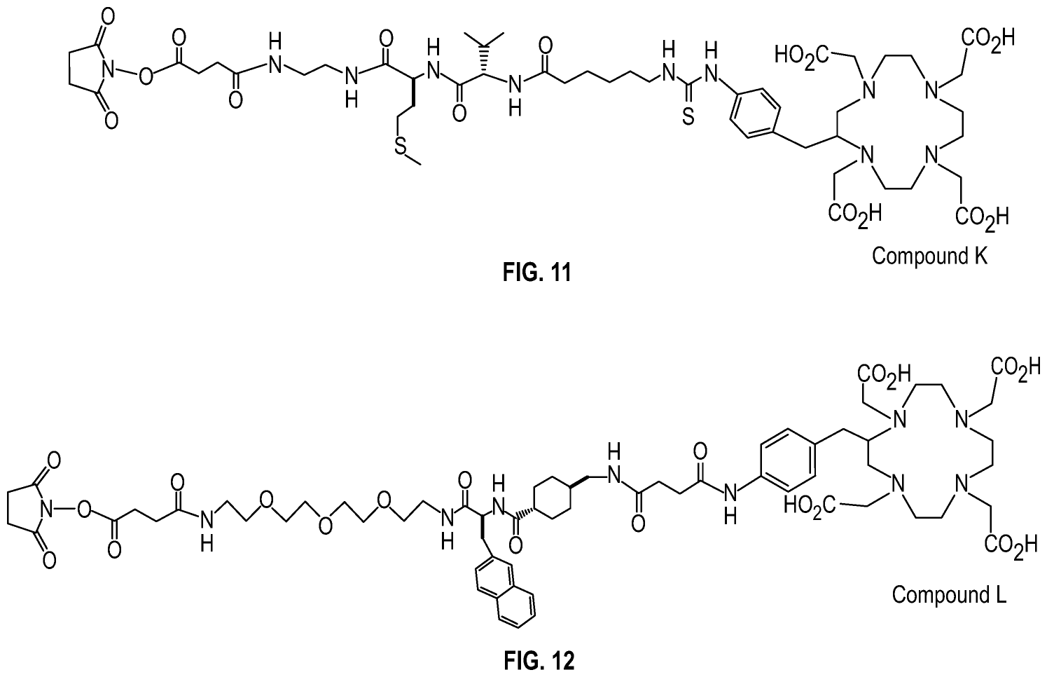

The bifunctional chelator compounds of the present disclosure may include a compound having the formula (K) (FIG. 11), or a metal complex thereof:

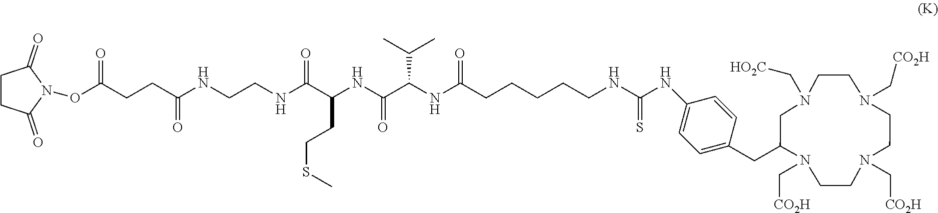 [SMILES: CSCC[C@H](NC(=O)[C@@H](NC(=O)CCCCCNC(=S)Nc1ccc(CC2CN(CC(=O)O)CCN(CC(=O)O)CCN(CC(=O)O)CCN2CC(=O)O)cc1)C(C)C)C(=O)NCCNC(=O)CCC(=O)ON1C(=O)CCC1=O]

The bifunctional chelator compounds of the present disclosure may include a compound having the formula (L) (FIG. 12), or a metal complex thereof:

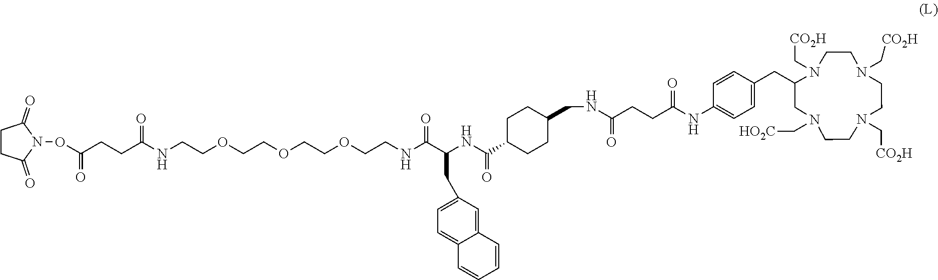 [SMILES: O=C(O)CN1CCN(CC(=O)O)CCN(CC(=O)O)C(Cc2ccc(NC(=O)CCC(=O)NC[C@H]3CC[C@H](C(=O)N[C@@H](Cc4ccc5ccccc5c4)C(=O)NCCOCCOCCOCCNC(=O)CCC(=O)ON4C(=O)CCC4=O)CC3)cc2)CN(CC(=O)O)CC1]

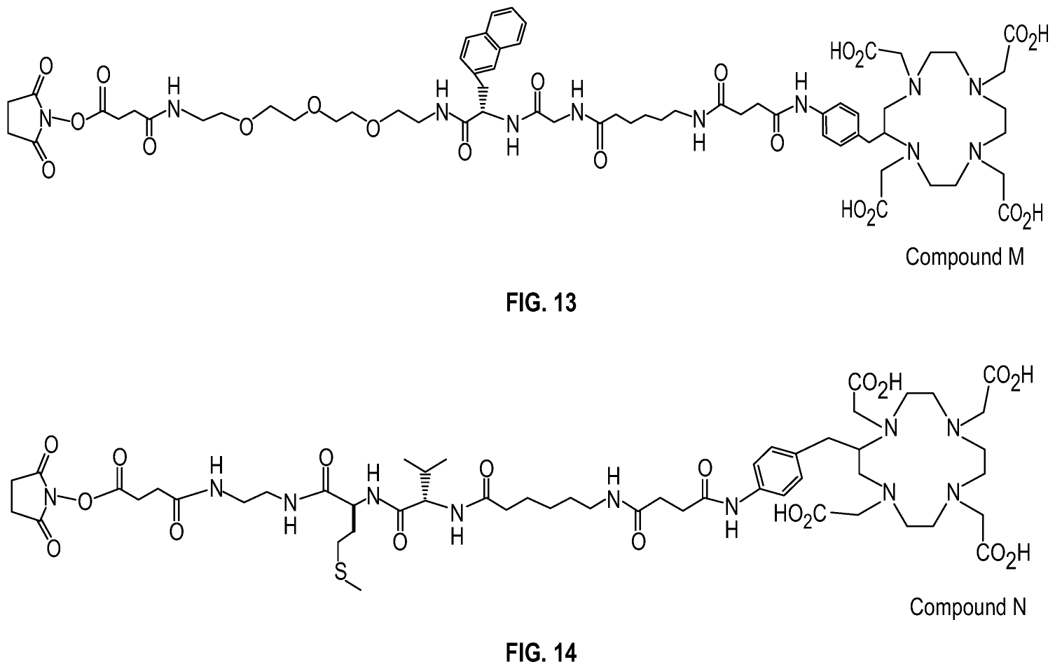

The bifunctional chelator compounds of the present disclosure may include a compound having the formula (M) (FIG. 13), or a metal complex thereof:

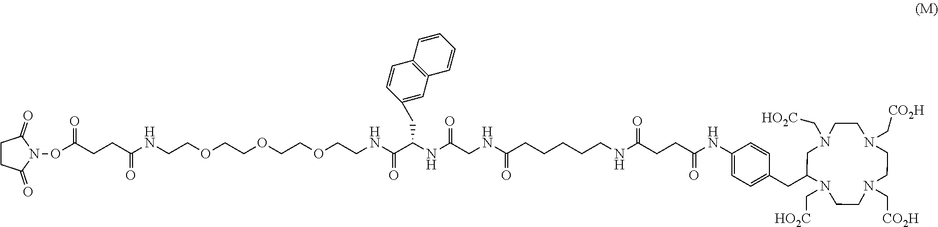 [SMILES: O=C(O)CN1CCN(CC(=O)O)CCN(CC(=O)O)C(Cc2ccc(NC(=O)CCC(=O)NCCCCCC(=O)NCC(=O)N[C@@H](Cc3ccc4ccccc4c3)C(=O)NCCOCCOCCOCCNC(=O)CCC(=O)ON3C(=O)CCC3=O)cc2)CN(CC(=O)O)CC1]

The bifunctional chelator compounds of the present disclosure may include a compound having the formula (N) (FIG. 14), or a metal complex thereof:

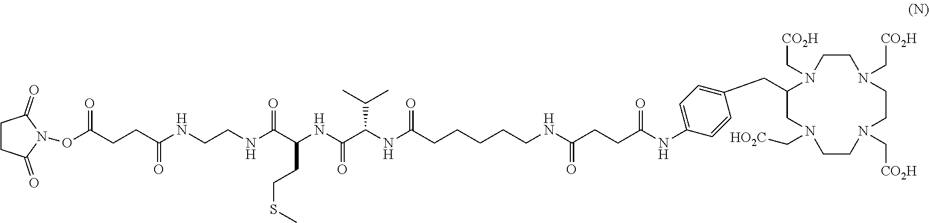 [SMILES: CSCC[C@H](NC(=O)[C@@H](NC(=O)CCCCCNC(=O)CCC(=O)Nc1ccc(CC2CN(CC(=O)O)CCN(CC(=O)O)CCN(CC(=O)O)CCN2CC(=O)O)cc1)C(C)C)C(=O)NCCNC(=O)CCC(=O)ON1C(=O)CCC1=O]

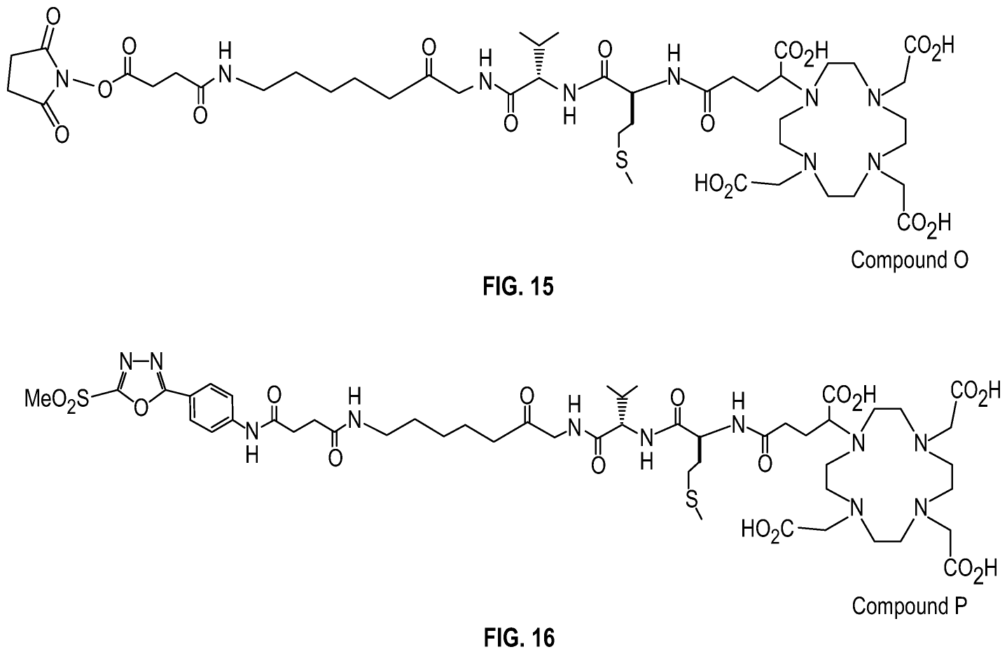

The bifunctional chelator compounds of the present disclosure may include a compound having the formula (O) (FIG. 15), or a metal complex thereof:

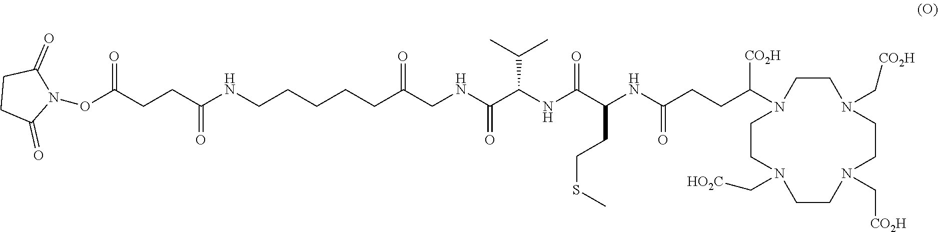 [SMILES: CSCC[C@H](NC(=O)CCC(C(=O)O)N1CCN(CC(=O)O)CCN(CC(=O)O)CCN(CC(=O)O)CC1)C(=O)N[C@H](C(=O)NCC(=O)CCCCCNC(=O)CCC(=O)ON1C(=O)CCC1=O)C(C)C]

The bifunctional chelator compounds of the present disclosure may include a compound having the formula (P) (FIG. 16), or a metal complex thereof:

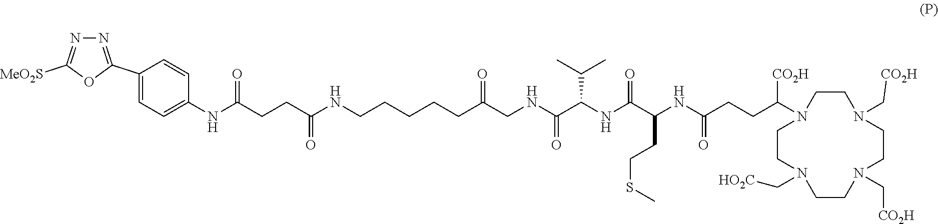 [SMILES: CSCC[C@H](NC(=O)CCC(C(=O)O)N1CCN(CC(=O)O)CCN(CC(=O)O)CCN(CC(=O)O)CC1)C(=O)N[C@H](C(=O)NCC(=O)CCCCCNC(=O)CCC(=O)Nc1ccc(-c2nnc(S(C)(=O)=O)o2)cc1)C(C)C]

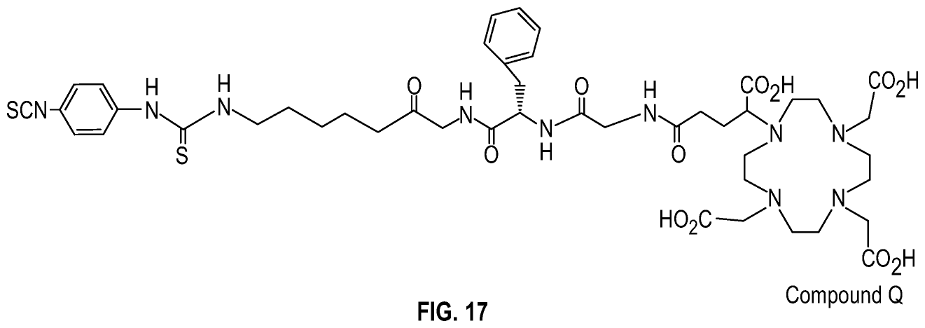

The bifunctional chelator compounds of the present disclosure may include a compound having the formula (Q) (FIG. 17), or a metal complex thereof:

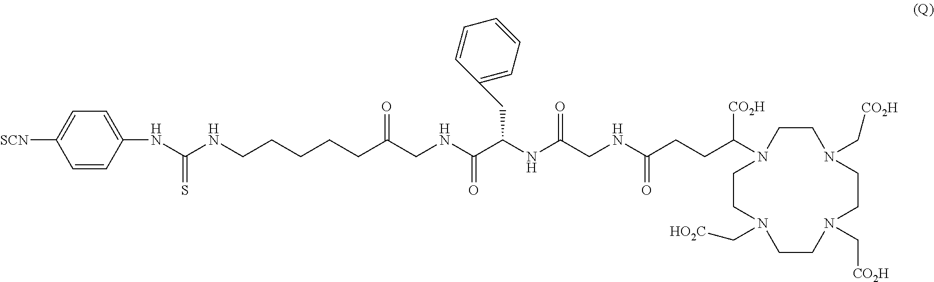

The bifunctional chelator compounds of the present disclosure may include a compound having the formula (I), or a metal complex thereof:

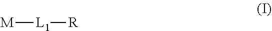

wherein R is a reactive group, L₁ is a linker group, and M is a chelator moiety.

The reactive group R may include any suitable reactive group for conjugation of the bifunctional chelator to a target molecule, such as a protein or a peptide. A reactive group may include, but is not limited to, N-hydroxysuccinimide ester/NHS ester (herein “NHS”), thiocyanate (hereinafter “SCN”), a phenyloxadiazolyl methylsulfone (hereinafter “PODS”), and the like. The present disclosure also provides the corresponding ethylsulfone and propylsulfone analogs of the phenyloxadiazolyl methylsulfone (PODS) bifunctional chelator compounds. The NHS or SCN reactive groups may be used for conjugation to molecules having primary amine groups. The PODS reactive group may be used for conjugation to molecules having free thiols. The present disclosure also provides corresponding bifunctional chelator compounds having, instead, a 4-pyridyl reactive group for conjugation to molecules including primary amine groups, such as via the Zincke reaction.

The chelator moiety M may include DOTA, DOTA-GA, DOTA derivative chelator moieties, or any of the chelator moieties disclosed herein.

The linker group L₁ may include at least one component selected from: a benzyl group, at least one amino acid and/or amino acid derivative, a PEG spacer, a non-aromatic cyclic hydrocarbon, a linear hydrocarbon, ethyleneamine, thiourea, and the like, wherein when more than one component is included, the reactive group is a terminal component and any additional components may be in any order.

L₁ may include the formula (IA):

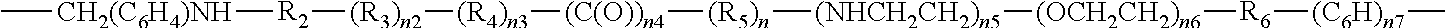

wherein n2-n7 is 0 or 1; R₂ is —C(S)—, —C(S)NH—, —C(O)CH₂CH₂—, C(O)CH₂CH₂C(O)—; R₃ is CH₂, NHCH₂, or (CH₂)ₙ₁, where n1 is 1 to 5; R₄ is a cyclic alkane having 5 to 8 carbons or linear alkane having 1 to 6 carbons; R₅ is an amino acid or amino acid derivative, wherein n is 1, 2, or 3; and R₆ is —NHC(S)NH—, —C(S)NH—, —C(O)NH—, NH(CH₂)₂NHC(O)(CH₂)₂, or NHC(O)(CH₂)₂—.

For Compound A, L₁ may include:

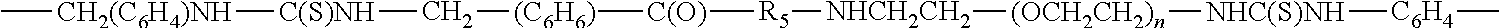

wherein R₅ is naphthylalanine.

For Compound B, L₁ may include:

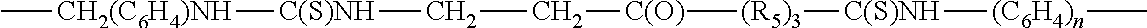

wherein R₅ is -methionine-valine-lysine-.

For Compound I, L₁ may include:

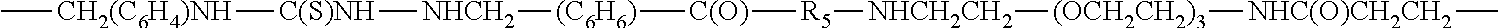

wherein R₅ is naphthylalanine.

For Compound J, L₁ may include:

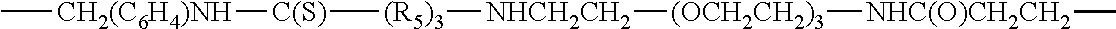

wherein R₅ is -naphthylalanine-glycyl-lysine-.

For Compound K, L₁ may include:

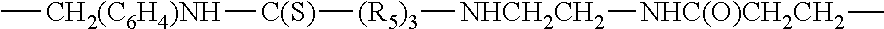

wherein R₅ is -methionine-valine-lysine-.

For Compound L, L₁ may include:

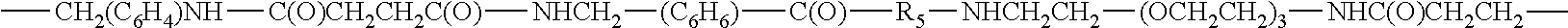

wherein R₅ is naphthylalanine.

For Compound M, L₁ may include:

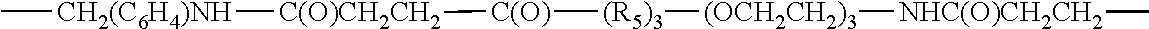

wherein R₅ is naphthylalanine-glycyl-lysine.

For Compound N, L₁ may include:

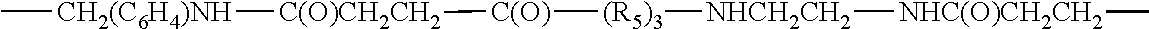

wherein R₅ is -methionine-valine-lysine-.

The bifunctional chelator compounds of the present disclosure may include a compound having the formula (II), or a metal complex thereof:

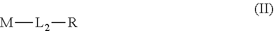

wherein R is a reactive group according to the present disclosure, L₂ is a linker group of the present disclosure, and M is a chelator moiety of the present disclosure.

The linker group L₂ may include the formula (IIA):

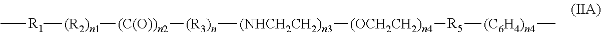

wherein: n1-n5 is 0 or 1; R₁ is —CH₂C(O)NHCH₂—, —C(CO₂H)CH₂CH₂C(O)NHCH₂—, —C(CO₂H)CH₂CH₂C(O)—, or —C(CO₂H)CH₂CH₂; R₂ is a cyclic alkane having 5 to 8 carbons or linear alkane having 1 to 6 carbons; R₃ is an amino acid or amino acid derivative; n is 1, 2, or 3; and R₄ is —NHC(S)NH—, —C(S)NH—, —NHC(O)CH₂CH₂C(O)NH—, —NHC(O)CH₂CH₂C(O)—, —NHC(O)CH₂CH₂—, —C(O)CH₂CH₂C(O)NH—, or —C(O)CH₂CH₂C(O)—.

For Compound C, L₂ may include:

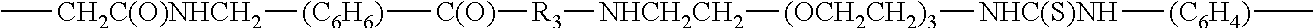

wherein R₃ is naphthylalanine.

For Compound D, L₂ may include:

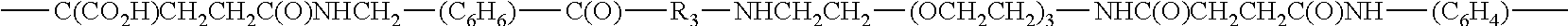

wherein R₃ is naphthylalanine.

For Compound E, L₂ may include:

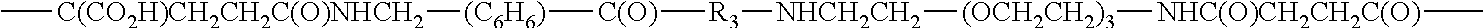

wherein R₃ is naphthylalanine.

For Compound F, L₂ may include:

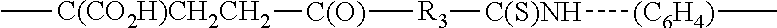

wherein R₃ is naphthylalanine-lysine.

For Compound G, L₂ may include:

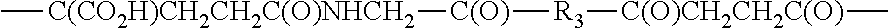

wherein R₃ is naphthylalanine-lysine.

For Compound H, L₂ may include:

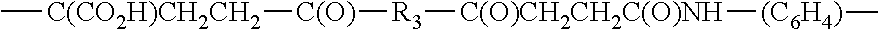

wherein R₃ is lysine-naphthylalanine-lysine.

For Compound O, L₂ may include:

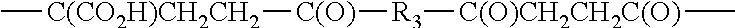

wherein R₃ is methionine-valine-lysine.

For Compound P, L₂ may include:

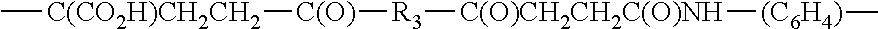

wherein R₃ is methionine-valine-lysine.

For Compound Q, L₂ may include:

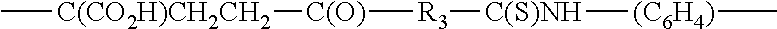

wherein R₃ is glycyl-phenyalanyl-lysine.

The bifunctional chelator compounds of the present disclosure may include a compound having the formula (III), or a metal complex thereof:

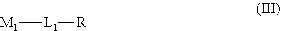

wherein R is a reactive group according to the present disclosure, Lₐ is a linker group of the present disclosure, and M₁ is a chelator moiety of the present disclosure.

A chelator moiety M₁ may include, but is not limited to, a chelator moiety having the formula (IIIA), (IIIB), or (IIIC):

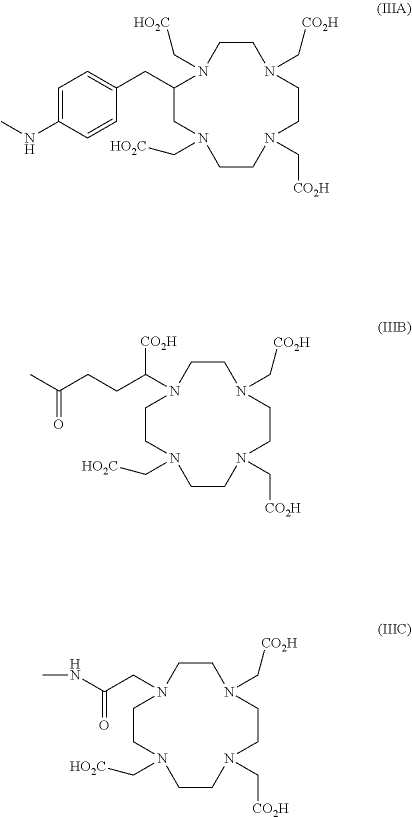 [SMILES: CNc1ccc(CC2CN(CC(=O)O)CCN(CC(=O)O)CCN(CC(=O)O)CCN2CC(=O)O)cc1 ; CC(=O)CCC(C(=O)O)N1CCN(CC(=O)O)CCN(CC(=O)O)CCN(CC(=O)O)CC1 ; CNC(=O)CN1CCN(CC(=O)O)CCN(CC(=O)O)CCN(CC(=O)O)CC1]

wherein IIIA is S-2-(4-aminobenzyl)-1,4,7,10-tetraazacyclododecane tetraacetic acid (i.e., p-NH₂-Bn-DOTA); IIIB is (R)-5-(tert-butoxy)-5-oxo-4-(4,7,10-tris(2-(tert-butoxy)-2-oxoethyl)-1,4,7,10-tetraazacyclododecan-1-yl)pentanoic acid (i.e., (R)-DOTA-GA(tBu)₄); and IIIC is 5-(tert-butoxy)-5-oxo-4-(4,7,10-tris(2-(tert-butoxy)-2-oxoethyl)-1,4,7,10-tetraazacyclododecane-1-yl)-methylamine.

A reactive group R may include, but is not limited to, a reactive group having the formula (IIID), (IIIE), or (IIIF):

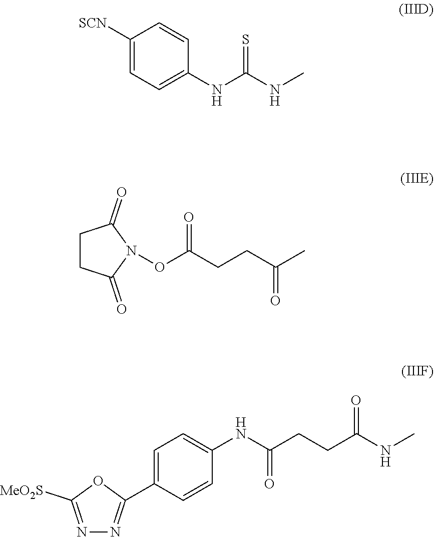

The linker group Lₐ may include the formula (IIIG):

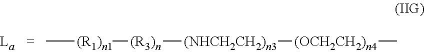

wherein: n1-n4 is 0 or 1; R₁ is —C(S)NHCH₂—(R₂)ₙ₂—C(O), —C(S)NHNHCH₂—(R₂)ₙ₂—C(O), —NHCH₂—(R₂)ₙ₂—C(O), —C(S)NH, —C(O)CH₂CH₂C(O)NH(R₂)C(O)—, —C(O)CH₂CH₂C(O)NHCH₂(R₂)C(O)—, —R₂C(O), or —C(O)CH₂CH₂C(O)NH—, wherein R₂ is a cyclohexane or CH₂; R₃ is an amino acid or amino acid derivative; and n is 1, 2, or 3.

Exemplary linker groups Lₐ include: —C(S)NHCH₂(C₆H₆)C(O)—R₃—(NHCH₂CH₂)—(OCH₂CH₂)₃—, wherein R₃ is naphthylalanine; —C(S)NHCH₂(CH₂)C(O)—R₃—, wherein R₃ is -methionine-valine-lysine-; —(C₆H₆)C(O)—R₃—(NHCH₂CH₂)—(OCH₂CH₂)₃—, wherein R₃ is naphthylalanine; —NHCH₂—(C₆H₆)—C(O)—R₃—(NHCH₂CH₂)—(OCH₂CH₂)₃—, wherein R₃ is naphthylalanine; —NHCH₂—(C₆H₆)—C(O)—R₃—(NHCH₂CH₂)—, wherein R₃ is naphthylalanine; —NHCH₂—C(O)—R₃—, wherein R₃ is naphthylalanine-lysine or glycyl-phenylalanyl-lysine; —R₃—, wherein R₃ is lysine-naphthylalanine-lysine or methionine-valine-lysine or glycyl-phenylalanyl-lysine; —C(S)NHNHCH₂(C₆H₆)C(O)—R₃—(NHCH₂CH₂)—(OCH₂CH₂)₃—, wherein R₃ is naphthylalanine; —C(S)NH—R₃—(NHCH₂CH₂)—(OCH₂CH₂)₃—, wherein R₃ is -naphthylalanine-glycyl-lysine-; —C(S)NH—R₃—(NHCH₂CH₂)—, wherein R₅ is -methionine-valine-lysine-; —C(O)CH₂CH₂C(O)NHCH₂(C₆H₆)C(O)—R₃—(OCH₂CH₂)₃—, wherein R₃ is naphthylalanine; —C(O)CH₂CH₂C(O)NH—R₃—(OCH₂CH₂)₃—, wherein R₃ is naphthylalanine-glycyl-lysine; and —C(O)CH₂CH₂C(O)NH—R₃—(NHCH₂CH₂)—, wherein R₃ is methionine-lysine-valine.

The bifunctional chelator compounds may include a PEG spacer, wherein a PEG spacer may include any inert spacer that does not affect the conformational properties of the compound to which the spacer is attached. A PEG spacer of the present disclosure may include, but is not limited to, a PEG linker having an average molecular weight from 0.1 KD to 50 KD. The average molecular weight may be at least 0.1 KD, including, but not limited to, at least 0.5, 1, 5, 10, 15, 20, 25, 30, 35, 40, 45, and at least 50 KD. The average molecular weight may be no more than 50 KD, including, but not limited to, 45, 40, 35, 30, 25, 20, 15, 10, 5, 1, 0.5, and 0.1 KD. Any combination of lower and upper limits may define the average molecular weight, including, but not limited to, 0.1 KD to 1 KD, 1 KD to 5 KD, 5 KD to 15 KD, 15 KD to 25 KD, 25 KD to 35 KD, and 35 KD to 50 KD. The PEG spacer of the present disclosure may include (PEG)ₙ, wherein n is 1 or an integer greater than 1, such as an integer from 1 to 1000 or any integer value therein or any subrange of integers therein, such as from 1 to 100 or 1 to 20. For example, n may be 1, 2, 3, 4, 5, 6, 7, 8, 9, or 10.

The bifunctional chelator compounds of the present disclosure may include at least one amino acid or amino acid derivative. The at least one amino acid or amino acid derivative, may include, but is not limited to, Naphthylalanine, lysine, valine, methionine, glycyl, phenylalanyl, and the like.

The bifunctional chelator compounds of the present disclosure may include a chelator moiety, which may include, but it not limited to, dodecane tetraacetic acid (hereinafter “DOTA”), DOTA-GA, DOTA derivative chelator moieties, and the like. As used herein, “GA” may refer to glutaric acid. The present disclosure provides bifunctional chelator compounds in which the chelator moiety includes 1,4,7,10-tetraazacyclododecane-1,4,7-triacetic acid (DO3A) or a derivative thereof; 1,4,7-triazacyclononane-1,4-diacetic acid (NODA) or a derivative thereof; 1,4,7-triazacyclononane-1,4,7-triacetic acid (NOTA) or a derivative thereof; 1,4,7,10-tetraazacyclododecane-1,4,7,10-tetraacetic acid (DOTA) or a derivative thereof; 1,4,7-triazacyclononane, 1-glutaric acid-4,7-diacetic acid (NODAGA) or a derivative thereof; 1,4,7,10-tetraazacyclodecane, 1-glutaric acid-4,7,10-triacetic acid (DOTAGA) or a derivative thereof; 1,4,8,11-tetraazacyclotetradecane-1,4,8,11-tetraacetic acid (TETA) or a derivative thereof; 1,4,8,11-tetraazabicyclo[6.6.2]hexadecane-4,11-diacetic acid (CB-TE2A) or a derivative thereof; diethylene triamine pentaacetic acid (DTPA), its diester, or a derivative thereof; 2-cyclohexyl diethylene triamine pentaacetic acid (CHX-A″-DTPA) or a derivative thereof; deforoxamine (DFO) or a derivative thereof; 1,2-[[6-carboxypyridin-2-yl]methylamino]ethane (H2dedpa) or a derivative thereof; DADA or a derivative thereof; 1,4,7,10-Tetraazacyclododecane-1,4,7,10-tetra(methylene phosphonic acid) (DOTP) or a derivative thereof; 4-amino-6-[[16-[(6-carboxypyridin-2-yl)methyl]-1,4,10,13-tetraoxa-7,16-diazacyclooctadec-7-yl]methyl]pyridine-2-carboxylic acid (MACROPA-NH2) or a derivative thereof; MACROPA or a derivative thereof; 1,4,7,10-tetrakis(carbamoylmethyl)-1,4,7,10-tetraazacyclododecane (TCMC) or a derivative thereof; {4-[2-(bis-carboxymethylamino)-ethyl]-7-carboxymethyl-[1,4,7]triazonan-1-yl}-acetic acid (NETA) or a derivative thereof; Diamsar or a derivative thereof; 1,4,7-triazacyclononane-1,4,7-tris[methyl(2-carboxyethyl)phosphinic acid (TRAP, PRP9, TRAP-Pr) or a derivative thereof; N,N′-bis(6-carboxy-2-pyridylmethyl)ethylenediamine-N,N′-diacetic acid (H4octapa) or a derivative thereof; N,N′-[1-benzyl-1,2,3-triazole-4-yl]methyl-N,N′-[6-(carboxy)pyridin-2-yl]-1,2-diaminoethane (H2azapa) or a derivative thereof; N,N″-[[6-(carboxy)pyridin-2-yl]methyl]diethylenetriamine-N,N′,N″-triacetic acid (H5decapa) or a derivative thereof; N,N′-bis(2-hydroxy-5-sulfobenzyl)ethylenediamine-N,N′-diacetic acid (SHBED) or a derivative thereof; N,N′-bis(2-hydroxybenzyl)ethylenediamine-N,N′-diacetic acid (HBED) or a derivative thereof; 3,6,9,15-tetraazabicyclo[9.3.1]pentadeca-1(15), 11,13-triene-3,6,9-triacetic acid (PCTA) or a derivative thereof; desferrioxamine B (DFO) or a derivative thereof; N,N′-(methylenephosphonate)-N,N′-[6-(methoxycarbonyl)pyridin-2-yl]methyl-1,2-diaminoethane (H6phospa) or a derivative thereof; 1,4,7,10,13,16-hexaazacyclohexadecane-N,N′,N″,N′″,N″″, N′″″-hexaacetic acid (HEHA) or a derivative thereof; 1,4,7,10,13-pentaazacyclopentadecane-N,N′,N″,N′″,N″″-pentaacetic acid (PEPA) or a derivative thereof; or 3,4,3-LI(1,2-HOPO) or a derivative thereof.

The protein or peptide may be a cancer cell targeting agent that binds to an antigen/target molecule that is preferentially expressed or overexpressed on mammalian cancer cells such as human cancer cells. The protein may be an antibody, such as a monoclonal antibody, or an antigen-binding fragment thereof such as a Fab fragment, Fab2 fragment, or scFv molecule, a minibody, a diabody, or a nanobody, or an antibody mimetic protein such as a designed ankyrin repeat proteins (DARPin).

The present disclosure provides compositions including, but not limited to, radiopharmaceutical compositions and/or radiodiagnostic (radio-imaging) compositions, and the like. Radiopharmaceutical compositions and/or radiodiagnostic compositions may include one or more pharmaceutically acceptable carriers or excipients. Such carriers and excipients are well known in the art. As a non-limiting example, injectable drug delivery systems include solutions, suspensions, gels, microspheres and polymeric injectables, and may include excipients such as solubility-altering agents (e.g., ethanol, propylene glycol and sucrose) and polymers (e.g., polycaprylactones and PLGA's). Radioconjugate aspects of the present disclosure may be formulated with excipients as substantially described in U.S. Pat. No. 10,420,851 or International Pub. No. WO 2017/155937, which are hereby incorporated by reference in their entirety. The formulation may include 0.5% to 5.0% (w/v) of one or more excipients, including, but not limited to, ascorbic acid, polyvinylpyrrolidone (PVP), human serum albumin (HSA), a water-soluble salt of HSA, any combination thereof, and the like. As a non-limiting example, the formulation may include 0.5-5% ascorbic acid; 0.5-4% polyvinylpyrrolidone (PVP); and a chelator conjugated affinity reagent protein such as an antibody, such as a monoclonal antibody, which may be radiolabeled or a chelated radionuclide in a buffered solution, such as in 50 mM PBS buffer, pH 7.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments thereof, such as Fab, di-Fab, as well as scFv molecules, diabodies, minibodies, single domain antibodies (sdAb), and nanobodies (VHH); (d) naturally occurring and non-naturally occurring, such as wholly synthetic antibodies, IgG-Fc-silent, and chimeric; and (e) bi-specific and multi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies may be human, humanized or nonhuman. When the present disclosure refers to or recites an “antibody,” it is intended as referring to any of the full-length antibodies or fragments thereof disclosed herein, unless explicitly denoted otherwise. Further, wherever in this disclosure specific antibodies are disclosed, aspects directed to antigen binding fragments of such antibodies, such as Fab or Fab₂ fragments, or corresponding scFv molecules are intended to be provided and disclosed. Similarly, wherever in this disclosure specific antibodies are disclosed, corresponding aspects directed to full-length antibodies, antigen-binding antibody fragments, such as Fab or Fab₂ fragments, or scFv molecules that have the same immunoglobulin heavy chain CDRs and/or immunoglobulin light chain CDRs are also intended to be provided and disclosed. Such corresponding aspects may include one or both of the heavy chain variable region amino acid sequence and the light chain variable region amino acid sequence of the recited reference antibody. Antibody heavy chain and light chain complementarity determining regions (CDRs) and regions may be defined/delineated according to the Kabat or IMGT numbering conventions. Further, wherever in this disclosure antibody heavy chain or light chain sequences are disclosed that include N-terminal leader sequences, corresponding aspects including or consisting of the respective light chains without its leader sequence, e.g., beginning instead with the first amino acid residue of its respective variable region, are also intended to be provided and disclosed.

Single chain Fv molecules (scFv) are single polypeptide chain antibody molecules that include an immunoglobulin light chain variable region domain (VL domain) and an immunoglobulin heavy chain variable region domain (VH domain) joined by a peptide linker (L). An scFv molecule represents either VL-L-VH if the VL domain is the N-terminal part of the scFv molecule or VH-L-VL if the VH domain is the N-terminal part of the scFv molecule. Methods for making scFv molecules and for designing suitable peptide linkers are disclosed in U.S. Pat. Nos. 4,704,692 and 4,946,778, where are hereby incorporated by reference in their entirety.

The present disclosure provides a conjugate of any of the bifunctional chelators disclosed herein and a protein, such as a scFv molecule or a protein including at least one scFv molecule segment, that may include, in consecutive order (from amino to carboxyl terminal end), amino acids 1-120 of an immunoglobulin heavy chain variable region as disclosed herein (as a non-limiting example, where amino acid residue 1 is the first residue of the heavy chain variable region according to the Kabat numbering convention), followed by a linker amino acid sequence, followed by the amino acid sequence of a light chain variable region disclosed herein. The sequence of either or both of the heavy chain variable region portion and the light chain variable region portion of the scFv molecule may be minus 1, 2, 3, 4, or 5 amino acids at one or both ends with respect to the full-length variable region sequence. Such conjugates may be radiolabeled by chelation of a radionuclide to the chelator moiety of the conjugate.

The scFv linker amino acid sequence may, for example, include any of the amino acid sequences:

(SEQ ID NO: 122)
KISGGGGSGGGGSGGGGSGGGGSGGGGSS,
(SEQ ID NO: 123)
SPNSASHSGSAPQTSSAPGSQ,

(G₃S)ₙ where, for example, n is 1, 2, 3, 4, 5, 6, 7, 8, 9, or 10, such as (G₃S)₄, or
(G₄S)ₙ where, for example, n is 1, 2, 3, 4, 5, 6, 7, 8, 9, or 10, such as (G₄S)₅.

It should be further understood herein that wherever in the sequences of this disclosure an immunoglobulin heavy chain sequence or an immunoglobulin light chain sequence are provided that may include an amino terminal signal peptide sequence, corresponding amino acid sequences without the signal peptide sequences are also intended to be disclosed and that the various aspects of the present disclosure may be embodied with either the versions of the proteins with the signal peptide sequence or corresponding versions without the signal peptide sequences, and in any combination.

A protein, such as an antibody or fragment thereof or scFv molecule, or an antibody mimetic or a peptide that is conjugated to any of the bifunctional linkers disclosed herein may specifically bind to target antigens specifically expressed by or overexpressed by various types of cancer cells, such as one or more cancer antigens, including, but not limited to, the human forms thereof: CD33, DR5, 5T4 (trophoblast glycoprotein), HER2 (ERBB2; Her2/neu), HER3, TROP2 (TROP-2, EGP1, EGP-1), mesothelin, TSHR, CD19, CD123, CD22, CD30, CD45, CD171, CD138, CS-1, CLL-1, GD2, GD3, B-cell maturation antigen (BCMA), Tn Ag, prostate specific membrane antigen (PSMA), ROR1, FLT3, fibroblast activation protein (FAP), a Somatostatin receptor, Somatostatin Receptor 2 (SSTR2), Somatostatin Receptor 5 (SSTR5), gastrin-releasing peptide receptor (GRPR), NKG2D ligands (such as MICA, MICB, RAET1E/ULBP4, RAET1G/ULBP5, RAET1H/ULBP2, RAET1/ULBP1, RAET1L/ULBP6, and RAET1N/ULBP3), LYPD3 (C4.4A), Nectin-4, urokinase plasminogen activator receptor (uPAR), Folate receptor alpha (FOLR1), CUB-domain containing protein 1 (CDCP1), Glypican-3 (GPC3), tenascin, tenascin-C, CEACAM5, Cadherin-3, CCK2R, Neurotensin receptor type 1 (NTSR1), human Kallikrein 2 (hK2), norepinephrine transporter, Integrin alpha-V-beta-6, CD37, CD66, CXCR4, Fibronectin extradomain B (EBD), LAT-1, Carbonic anhydrase IX (CAIX), B7-H3 (a/k/a CD276), Disialoganglioside GD2 Antigen (GD2), calreticulin, phosphatidylserine, GRP78 (BiP), TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (Fra), MUC1, epidermal growth factor receptor (EGFR), EGFRVIII, NCAM, Prostase, PAP, ELF2M, Ephrin B2, IGF-I receptor, CAIX, LMP2, gplOO, ber-abl, tyrosinase, EphA2, Fucosyl GM1, sLc, GM3, DR5, 5T4, TGS5, HMWMAA, o-acetyl-GD2, Folate receptor beta, TEMI/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6,E7, MAGE A1, MAGEA3, MAGEA3/A6, ETV6-AML, sperm protein 17, XAGEI, Tie 2, MAD-CT-1, MAD-CT-2, Fos-related antigen 1, prostein, survivin and telomerase, PCTA-I/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYPIB 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, GPA7, IGLL1, FGFR2, FGFR2b, Six-transmembrane epithelial antigen of prostate 1 (STEAP1), MUC17, claudin-18 isoform 2 (CLDN18.2), and Sortilin (Neurotensin receptor-3). Such conjugates radiolabeled by chelation with a radionuclide may be used to treat and/or diagnostically image a cancer in a mammalian subject such as a human patient. Targeting agents that are conjugated with a bifunctional chelator as disclosed herein may also include those that bind antigens expressed by immunosuppressive cells found in or associated with cancers such as CCR8 antigen expressed by tumor associated regulatory T-cells (Tregs) and CD33 antigen expressed by myeloid-derived suppressor cells (MDSCs) and tumor-associated macrophages (TAMs).

CD38 targeting agents that may be conjugated with any of the bifunctional chelators disclosed herein and radiolabeled for use as a radioconjugate include the monoclonal antibodies daratumumab (Darzalex®, Janssen Biotech, Inc.; heavy chain (SEQ ID NO:124), CDR-H1 (SEQ ID NO:125), CDR-H₂ (SEQ ID NO:126), CDR-H₃ (SEQ ID NO:127), light chain (SEQ ID NO:128), CDR-L1 (SEQ ID NO: 129), CDR-L₂ (SEQ ID NO:130) and CDR-L₃ (SEQ ID NO:131)), isatuximab (Sarclisa®, a/k/a SAR650984, Sanofi; reported heavy chain SEQ ID NO:132, CDR-H1 (SEQ ID NO:133), CDR-H₂ (SEQ ID NO:134), CDR-H₃ (SEQ ID NO:135), reported light chain SEQ ID NO: 136, CDR-L1 (SEQ ID NO:137), CDR-L₂ (SEQ ID NO:138), CDR-L₃ (SEQ ID NO:139)), and MOR202 (MorphoSys AG; U.S. Pat. No. 8,088,896), as well as CD38-binding fragments thereof and antibodies having the same CDRs as daratumumab, isatuximab, or MOR202. CD38 targeting agents that may be conjugated with any of the bifunctional chelators disclosed herein and radiolabeled for use as a radioconjugate may include, but are not limited to, any of the anti-CD38 monoclonal antibodies disclosed in any of U.S. Pat. Nos. 8,088,896, 7,829,673, Int'l Pub. No. WO 2008/047242, U.S. Pat. No. 8,153,765 and U.S. Pub. No. 20210171653, CD38-binding fragments of any of said antibodies, and antibodies including the same CDRs as said antibodies.

The present disclosure provides a conjugate of any of the bifunctional chelators disclosed herein with a protein, such as an antibody, immunoglobulin heavy chain, immunoglobulin light chain, antibody fragment such as Fab fragment, Fab2 fragment or scFv molecules or fusion proteins, that include any of the amino acid sequences or combinations thereof set forth in SEQ ID NOS: 1-139. The present disclosure provides a conjugate of any of the bifunctional chelators disclosed herein with any of the proteins, including, but not limited to, the antibodies, immunoglobulin heavy chains, immunoglobulin light chains, antibody fragments, such as Fab fragments, Fab2 fragments or scFv molecules, or fusion proteins that recognize cancer- or cancer-related antigens, disclosed in International Pub. No. WO2022235676 (of Int'l App. No. PCT/US2022/027479), which is hereby incorporated by reference in its entirety.

The protein that is conjugated with any of the bifunctional chelators disclosed herein may also be a modification of any one of the sequences set forth in any SEQ ID NOS: 1-139 that comprises one or more amino acid substitutions that have little to no effect on the structure or function of the sequence (e.g., conservative substitutions). In some aspects, two sequences are considered to be “homologous” to one another if the percent amino acid sequence identity is at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%.

DR5 targeting agents that may be conjugated with any of the bifunctional chelators disclosed herein and radiolabeled for use as a radioconjugate, may include, but are not limited to, any one or more of the monoclonal anti-DR5 antibodies mapatumumab, conatumumab, lexatumumab, tigatuzumab, drozitumab, and LBY-135.

5T4 targeting agents that may be conjugated with any of the bifunctional chelators disclosed herein and radiolabeled for use as a radioconjugate may include, but are not limited to, any one or more of the monoclonal anti-5T4 antibodies MED10641, ALG.APV-527, Tb535, H₆-DM5, and ZV0508.

HER2 targeting agents that may be conjugated with any of the bifunctional chelators disclosed herein and radiolabeled for use as a radioconjugate may include the monoclonal antibodies trastuzumab and pertuzumab. The amino acid sequences of the light chain and the heavy chain of Trastuzumab reported by DrugBank Online are: light chain (SEQ ID NO:86) and heavy chain (SEQ ID NO:87). The amino acid sequences of the light chain and the heavy chain of Pertuzumab reported by DrugBank Online are: light chain (SEQ ID NO:88) and heavy chain (SEQ ID NO:89). The HER2 targeting agent used may be the ADC fam-trastuzumab deruxtecan-nxki (Enhertu®; Daiichi Sankyo, Japan).

HER3 targeting agents that may be conjugated with any of the bifunctional chelators disclosed herein and radiolabeled for use as a radioconjugate may include any one or more of the monoclonal antibodies patritumab, seribantumab, lumretuzumab, elgemtumab, GSK2849330, or AV-203 (Avco Oncology) or any of the anti-HER3 antibodies disclosed in U.S. Pat. Nos. 10,494,441; 9,828,635; or U.S. Pub. No. 20210025006. An HER3 antibody may include, but is not limited to, an immunoglobulin heavy chain variable region including a CDRH1 including SEQ ID NO:56, a CDRH2 including SEQ ID NO:57, and a CDRH3 including SEQ ID NO:58, an immunoglobulin light chain variable region including a CDRL1 including SEQ ID NO:59, a CDRL2 including SEQ ID NO:60, and a CDRL3 including SEQ ID NO:61. An exemplary HER3 antibody includes an immunoglobulin heavy chain variable region including SEQ ID NO:62 and/or an immunoglobulin light chain variable region including SEQ ID NO:63. An exemplary HER3 antibody includes an immunoglobulin heavy chain amino acid sequence of SEQ ID NO:64 and/or an immunoglobulin light chain amino acid sequence of SEQ ID NO:65. The HER3 targeting agent used may be the ADC patritumab deruxtecan.

TROP2 targeting agents that may be conjugated with any of the bifunctional chelators disclosed herein and radiolabeled for use as a radioconjugate may include, but are not limited to, the monoclonal antibodies Sacituzumab and Datopotamab, antibodies having one or both of the heavy chain and light chain of said antibodies, antibodies having one or both of the heavy chain variable region and the light chain variable region of said antibodies, and antibodies having one or both of the heavy chain CDRs and the light chain CDRs of said antibodies, or TROP2-binding fragments of any of the aforementioned antibodies, such as Fab fragments, Fab₂ fragments, or corresponding scFv molecules. Sacituzumab biosimilar is commercially available as Catalog No. A2175 from BioVision Incorporated (an Abcam company, Waltham, MA, USA). Datopotamab biosimilar is commercially available as Catalog No. PX-TA1653 from ProtcoGenix (Schiltigheim, France). The TROP2 targeting agent used may be the ADC Sacituzumab govitecan-hziy (Trodelvy®, Gilead Sciences, Inc., Foster City, CA, USA).

Further TROP2 targeting agents that may be conjugated with any of the bifunctional chelators disclosed herein and radiolabeled for use as a radioconjugate may include a monoclonal antibody having a heavy chain SEQ ID NO:66 and/or a light chain SEQ ID NO:71 (reported as the heavy and light chains of Sacituzumab), or an antibody including one or both of the heavy chain variable region (SEQ ID NO:67) or the light chain variable region (SEQ ID NO:72) of said chains, or an antibody including 1, 2, or 3 of the heavy chain CDRs of said heavy chain (CDR H1-3: SEQ ID NOS:68-70 respectively) and/or 1, 2 or 3 of the light chain CDRs of said light chain (CDR L1-3: SEQ ID NOS:73-75 respectively), and any of the anti-human TROP antibodies disclosed in U.S. Pat. No. 7,238,785 (such as mAb hRS7), U.S. Pat. Nos. 9,492,566, 10,195,517, or U.S. Pat. No. 11,116,846, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

TROP2 targeting agents that may be conjugated with any of the bifunctional chelators disclosed herein and radiolabeled for use as a radioconjugate may include, but are not limited to, a monoclonal antibody heavy chain SEQ ID NO:76 and/or a light chain SEQ ID NO:81 (reported as the heavy and light chains of Datopotamab), or an antibody including one or both of the variable region of said heavy chain (SEQ ID NO:77) and the variable region of said light chain (SEQ ID NO:82), or an antibody including 1, 2, or 3 of the heavy chain CDRs of said heavy chain (CDRs 1-3: SEQ ID NOS:78-80 respectively) and/or 1, 2 or 3 of the light chain CDRs of the said light chain (CDR H1-3: SEQ ID NOS:83-85 respectively), and any of the anti-human TROP antibodies disclosed in Int'l Pub. No. WO2015098099 or U.S. Pub. No. 20210238303, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

CD33 targeting agents that may be conjugated with any of the bifunctional chelators disclosed herein and radiolabeled for use as a radioconjugate may include the monoclonal antibodies lintuzumab (HuM195), gemtuzumab, and vadastuximab.

MUC1 targeting agents that may be conjugated with any of the bifunctional chelators disclosed herein and radiolabeled for use as a radioconjugate include hTAB004 (OncoTAb, Inc.) and any of the anti-MUC1 antibodies or antibody fragments disclosed in any of U.S. Pub. No. 20200061216 and U.S. Pat. Nos. 8,518,405; 9,090,698; 9,217,038; 9,546,217; 10,017,580; 10,507,251 10,517,966; 10,919,973; 11,136,410; and 11,161,911.

LYPD3 (C4.4A) targeting agents that may be conjugated with any of the bifunctional chelators disclosed herein and radiolabeled for use as a radioconjugate may include, but are not limited to, BAY 1129980 (a/k/a Lupartumab amadotin; Bayer AG, Germany) an Auristatin-based anti-C4.4A (LYPD3) ADC or its antibody component Lupartumab, IgG1 mAb GT-002 (Glycotopc GmbH, Germany) and any of those disclosed in U.S. Pub. No. 20210309711, 20210238292, 20210164985, 20180031566, 20170158775, or 20150030618, 20120321619, Canadian Patent Application No. CA3124332A1, Taiwan Application No. TW202202521A, or Int'l Pub. No. WO2021260208, WO2007044756, WO2022042690, or WO2020138489.

5T4 targeting agents that may be conjugated with any of the bifunctional chelators disclosed herein and radiolabeled for use as a radioconjugate may include, but are not limited to, 5T4-binding, single chain, chimeric antibody-superantigen fusion proteins such as C₂₁₅Fab-SEA (SEQ ID NO:93), 5T4Fab-SEA_{D227A} (SEQ ID NO:94), and 5T4Fab-SEA/E-120 (SEQ ID NO:95). For example, naptumomab estafenatox is SEQ ID NO:96 (chimeric heavy chain component) non-covalently bound to SEQ ID NO:97 (light chain component). The heavy chain may include a 5T4 Fab heavy chain component (corresponding to residues 1 to 222 of SEQ ID NO:95), the SEA/E-120 superantigen (corresponding to residues 226 to 458 of SEQ ID NO:95), and a GGP tripeptide linker (corresponding to residues 223-225 of SEQ ID NO:95) covalently linking the Fab heavy chain and SEA/E-120 components. The light chain may include residues 459 to 672 of SEQ ID NO:95.

5T4 targeting agents that may be conjugated with any of the bifunctional chelators disclosed herein and radiolabeled for use as a radioconjugate may include, but are not limited to, C215Fab-SEA (SEQ ID NO:93), 5T4Fab-SEAD227A (SEQ ID NO:94), 5T4Fab-SEA/E-120 (SEQ ID NO:95); SEQ ID NO:96, Naptumomab estafenatox reported as SEQ ID NO:96 (heavy chain component) non-covalently associated with SEQ ID NO:97 (light chain component), an antibody including the Fab component of Naptumomab estafenatox, an antibody including the heavy chain component of any one of SEQ ID NOS:93-96 (for example, lacking the enterotoxin components thereof), a 5T4-binding antibody including the heavy chain component of any one of SEQ ID NOS:93-96 and an associated light chain component such as the light chain component of SEQ ID NO:97, a 5T4-binding antibody (such as a single- or multi-chain antibody, such as but not limited to a human or humanized IgG, such as IgG1) including a heavy chain component that includes 2 or 3 of the heavy chain CDRs present in any one of SEQ ID NOS:93-96, Naptumomab estafenatox modified by covalent linkage of the heavy chain and light chain components to each other by disulfide bonds (between cysteine residues in the manner of a Fab) and/or by a bifunctional thiol reactive crosslinking agent which may include and introduce a chelating moiety (such as DOTA), any of the chimeric 5T4-binding superantigen fusion proteins of U.S. Pat. Nos. 7,615,225, 10,314,910 and U.S. Pub. No. 20200101160, any of the preceding 5T4 targeting agents in which the antibody component(s) is/are humanized, any of the preceding in which an amino acid (for example, of a heavy chain and/or of a light chain) is substituted to a cysteine, and any of the preceding further including at least one additional amino acid (for example, in a heavy chain and/or in a light chain thereof) of which at least one is a cysteine and/or at least one is a lysine (for example, one or more additional C-terminal amino acids of which one or more are cysteines and/or of which one or more are lysines).

Light chain variations that may also be used include, but are not limited to, SEQ ID NO:99 (SEQ ID NO:97 with C-terminal lysine), SEQ ID NO: 100 (SEQ ID NO: 97 with C-terminal cysteine), SEQ ID NO: 101 (SEQ ID NO:97 with C-terminal lys-cys), and a light chain or light chain segment including 2 or 3 of the CDRs of the light chain of SEQ ID NO:97—namely CDR-L1 KASQSVSNDVA (SEQ ID NO:119), CDR-L2 YTSSRYA (SEQ ID NO:120), and CDR-L3 QQDYNSPPT (SEQ ID NO:121).

Further 5T4 targeting agents that may be conjugated with any of the bifunctional chelators disclosed herein, and optionally radiolabeled, include proteins having or consisting of the following:
SEQ ID NO:102 [heavy chain portion only of SEQ ID NO:96];
an immunoglobulin heavy chain or heavy chain segment including 2 or 3 of the heavy chain CDRs found in SEQ ID NO:20, namely CDR-H1 GYYMH (SEQ ID NO:116), CDR-H2 RINPNNGVTLYNQKFKD (SEQ ID NO:117), and CDR-H3 STMITNYVMDY (SEQ ID NO:118);
SEQ ID NO: 103 [SEQ ID NO:96 including linker, excluding enterotoxin portion];
SEQ ID NO: 104 [SEQ ID NO:102 with C-terminal lysine];
SEQ ID NO:105 [SEQ ID NO: 103 with C-terminal lysine];
SEQ ID NO: 106 [SEQ ID NO:102 with C-terminal cysteine];
SEQ ID NO:107 [SEQ ID NO:103 with C-terminal cysteine];
SEQ ID NO:108 [SEQ ID NO: 102 with C-terminal lys-cys];
SEQ ID NO: 109 [SEQ ID NO: 103 with C-terminal lys-cys];
SEQ ID NO: 110 [5T4Fab-SEA/E-120 (SEQ ID NO:95) with C-terminal lysine];
SEQ ID NO:111 [SEQ ID NO:96 with C-terminal lysine];
SEQ ID NO:112 [5T4Fab-SEA/E-120 (SEQ ID NO:95) with C-terminal cysteine];
SEQ ID NO:113 [SEQ ID NO:96 with C-terminal cysteine];
SEQ ID NO:114 [5T4Fab-SEA/E-120 (SEQ ID NO:95) with C-terminal lys-cys];
SEQ ID NO:115 [SEQ ID NO:96 with C-terminal lys-cys]; and
any of SEQ ID NOS:96, 102-109, 111, 113 and 115 in non-covalent association with any of light chain SEQ ID NOS:97 and 99-101 to form a Fab without covalent cross-linking between chains, or a disulfide-bonded (cross-linked) form of such a Fab, or a form of such a Fab covalently cross-linked by a bifunctional cross-linker that may include a chelator such as DOTA.

The 5T4 targeting agent that may be conjugated to any of the bifunctional chelators disclosed herein may include, but is not limited to, an antibody, an antigen-binding antibody fragment, such as Fab or Fab₂, or an scFv molecule or a fusion protein, that includes immunoglobulin heavy chain CDRs 1, 2 and 3 as set forth in SEQ ID NOS: 116, 117 and 118 respectively, and/or immunoglobulin light chain CDRs 1, 2 and 3 as set forth in SEQ ID NOS:119, 120 and 121 respectively. Such 5T4 targeting agents may further include an enterotoxin portion as disclosed herein, such as any of the modified enterotoxins SEA/E-120 (SEQ ID NO:91), SEAD227A (SEQ ID NO:92), and SEQ ID NO:98.

Where the 5T4 targeting agent includes a heavy chain and a light chain, one or both of the heavy chain and the light chain may be radiolabeled, for example, by chemical conjugation to any of the bifunctional chelators disclosed herein, and chelation of a radionuclide, such as ²²⁵Ac or ¹⁷⁷Lu, by the chelator.

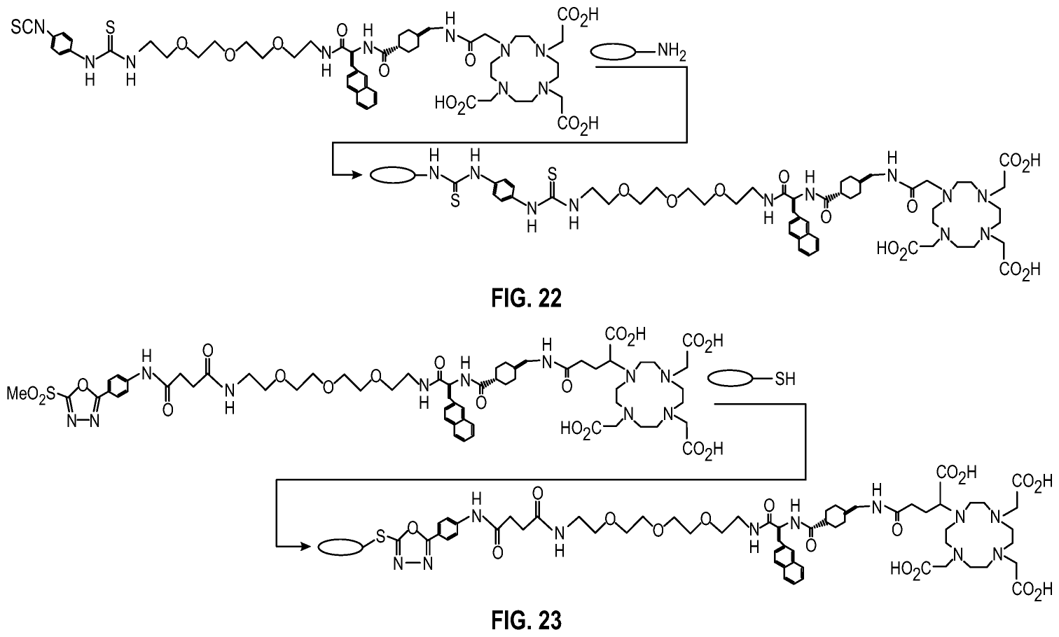

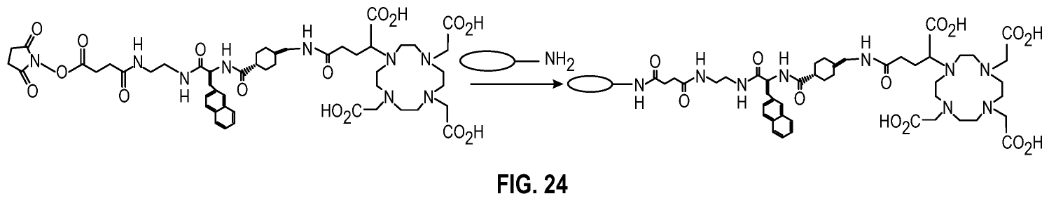

The present disclosure provides a method for manufacturing a chelator-conjugated molecule, including the steps: reacting a molecule having at least one primary amine group with a bifunctional chelator compound of the present disclosure having an NHS or SCN reactive group to conjugate a chelator moiety of the bifunctional chelator compound to the molecule and/or reacting a molecule having at least one free thiol group with a bifunctional chelator compound of the present disclosure having a PODS reactive group to conjugate the bifunctional chelator compound to the molecule. Exemplary conjugation reactions are shown in FIGS. 22-24. The molecule may include, but is not limited to, a peptide, a synthetic peptide, a protein, a peptide ligand of a receptor protein, or a recombinant protein. The molecule may include, but is not limited to, an antibody, a monoclonal antibody, an antigen-binding fragment of an antibody, an antigen-binding fragment of a monoclonal antibody, an immunoglobulin heavy chain, an immunoglobulin heavy chain variable region, an immunoglobulin light chain, an immunoglobulin light chain variable region, a nanobody, a scFv molecule, or an antibody mimetic protein.

The method may include chelating one or more radionuclides to the chelator moiety of the bifunctional chelator compound before and/or after performing the conjugation. One or more radionuclide includes, but is not limited to, one or more of ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, and ²²⁷Th. Accordingly, the molecule may include, but is not limited to, a 5T4 targeting agent of the present disclosure, wherein the 5T4 targeting agent binds human 5T4, such as naptumomab estafenatox. The molecule may include, but is not limited to, a CD38 targeting agent of the present disclosure.

The 5T4 targeting agent may include, but is not limited to, an antibody, an antibody fragment or an scFv molecule including an immunoglobulin heavy chain having complementarity determining regions 1-3 with amino acid sequences as set forth in SEQ ID NOS:116-118, respectively, and/or immunoglobulin light chain having complementarity determining regions 1-3 with amino acid sequences as set forth in SEQ ID NOS: 119-121, respectively.

The present disclosure further provides a method of manufacturing a chelator-conjugated molecule according to Example 1.

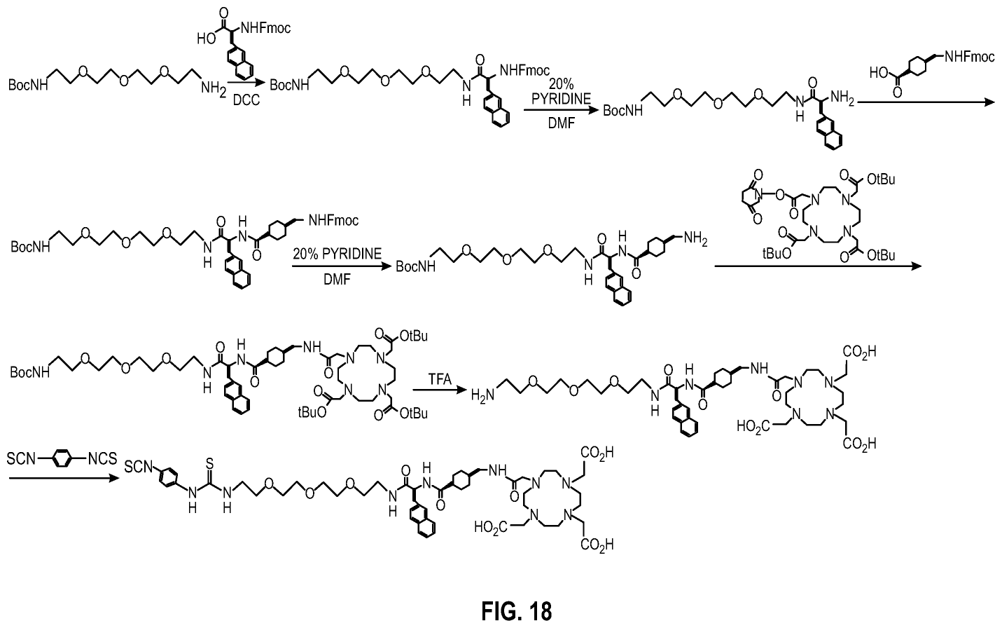

The present disclosure provides a method of synthesizing a bifunctional chelator compound of the present disclosure according to Example 2, as shown in FIGS. 18-21.

### Definitions

Unless otherwise defined, all terms (including technical and scientific terms) used herein have the same meaning as commonly understood by one of ordinary skill in the art to which this disclosure belongs.

As used herein, the term “and/or” includes any and all combinations of one or more of the associated listed items. Likewise, as used in the following detailed description, the term “or” is intended to mean an inclusive “or” rather than an exclusive “or.” That is, unless specified otherwise, or clear from context, “X employs A or B” is intended to mean any of the natural inclusive permutations. Thus, if X employs A; X employs B; or X employs both A and B, then “X employs A or B” is satisfied under any of the foregoing instances.

The terminology used herein is for the purpose of describing particular examples only and is not intended to be limiting. As used herein, the singular forms “a”, “an”, and “the” may be intended to include the plural forms as well, unless the context clearly dictates otherwise. As example, “a” compound may comprise one or more compounds, and the like.

The terms “comprises”, “comprising”, “including”, “having”, and “characterized by”, may be inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms may be to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics may be excluded from such an embodiment, but any compositions, materials, components, elements, features, integers, operations, and/or process steps that do not materially affect the basic and novel characteristics may be included in the embodiment.

Any method steps, processes, and operations described herein may not be construed as necessarily requiring their performance in the particular order discussed or illustrated, unless specifically identified as an order of performance. Thus, where a particular order is exemplified in this disclosure, corresponding aspects or embodiments having different ordering are also intended to be provided and disclosed. It is also understood that additional or alternative steps may be employed, unless otherwise indicated.

In addition, features described with respect to certain example embodiments may be combined in or with various other example embodiments in any permutational or combinatory manner. Different aspects or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included may be combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

In the description, certain details are set forth in order to provide a better understanding of various aspects of the systems and methods disclosed herein. However, one skilled in the art will understand that these aspects may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various embodiments.

While specific aspects of the disclosure have been provided hereinabove, the disclosure may, however, be embodied in many different forms and should not be construed as necessarily being limited to only the embodiments disclosed herein. Rather, these embodiments may be provided so that this disclosure is thorough and complete, and fully conveys various concepts of this disclosure to skilled artisans.

All numerical quantities stated herein may be approximate, unless stated otherwise. Accordingly, the term “about” may be inferred when not expressly stated. The numerical quantities disclosed herein may be to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the term “about” refers to values within an order of magnitude, potentially within 5-fold or 2-fold of a given value. Notwithstanding the approximations of numerical quantities stated herein, the numerical quantities described in specific examples of actual measured values may be reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges may be continuous and include every value between the minimum and maximum values. Any maximum numerical limitation recited herein is intended to include all lower numerical limitations. Any minimum numerical limitation recited herein is intended to include all higher numerical limitations.

Features or functionality described with respect to certain example aspects may be combined and sub-combined in and/or with various other example aspects. Also, different aspects and/or elements of example aspects, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, may be at least partially performed via at least one entity or actor in any manner.

All documents cited herein may be incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular aspects have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications, and substitutions. This application including the appended claims is therefore intended to cover all such changes and modifications that may be within the scope of this application.

The term “functionally equivalent variant” or “functionally active variant” of an amino acid sequence of the present disclosure refers to a sequence resulting from modification of the amino acid sequence of the present disclosure by insertion, deletion, or substitution of one or more amino acids or nucleotides within the sequence or at either or both distal ends of the sequence, and which modification does not affect (in particular impair) a function of the amino acid sequence. The functionally active variant may be obtained by sequence alterations in the amino acid sequence, wherein the sequence alterations retain a function of the unaltered amino acid sequence. Such sequence alterations may include, but are not limited to, (conservative) substitutions, additions, deletions, mutations, and insertions.

The variant of the peptide is functionally active in the context of this disclosure if the activity of the amino acid sequence amounts to at least 10%, preferably at least 25%, more preferably at least more preferably 50%, even more preferably at least 70%, still more preferably at least 80%, especially at least 90%, particularly at least 95%, and most preferably at least 99% of the biological activity of the amino acid sequence as used according to the present disclosure, including the amino acid sequence without sequence alteration (i.e. the original sequence).

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with unchanged polar side chains, with small side chains, with large side chains, etc.

In another aspect of the present disclosure, the amino acid sequences as defined above may be modified by a variety of chemical techniques to produce derivatives having essentially the same activity (as defined above for fragments and variants) as the amino acid sequences, and optionally having other desirable properties.

As used herein, “percent (%) amino acid sequence identity” with respect to the amino acid sequences identified herein is defined as the percentage of amino acid residues in a candidate sequence that are identical with the amino acid residues in the specific amino acid sequence, after aligning the sequence and introducing gaps, if necessary, to achieve the maximum percent sequence identity, and not considering any conservative substitutions as part of the sequence identity. Those skilled in the art may determine appropriate parameters for measuring alignment, including any algorithms such as BLAST needed to achieve maximal alignment over the full length of the sequences being compared.

### Aspects

Aspect 1: A bifunctional chelator compound comprising a formula (A)

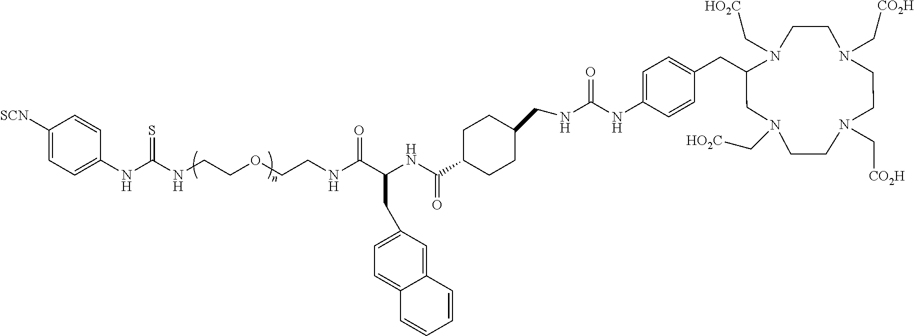

wherein n is 1 or an integer greater than 1.

Aspect 2: The bifunctional chelator of aspect 1, further comprising at least one radionuclide, wherein the radionuclide is chelated by the bifunctional chelator.

Aspect 3: The bifunctional chelator compound according to any of the foregoing aspects, wherein the at least one radionuclide comprises ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, or ²²⁷Th.

Aspect 4: A bifunctional chelator compound comprising a formula (B)

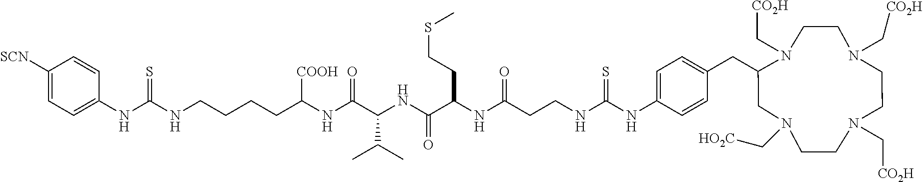

Aspect 5: The bifunctional chelator of aspect 4, further comprising at least one radionuclide, wherein the radionuclide is chelated by the bifunctional chelator.

Aspect 6: The bifunctional chelator compound according to any of the foregoing aspects, wherein the at least one radionuclide comprises ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, or ²²⁷Th.

Aspect 7: A bifunctional chelator compound comprising a formula (C) through formula (Q).

Aspect 8: The bifunctional chelator of aspect 7, further comprising at least one radionuclide, wherein the radionuclide is chelated by the bifunctional chelator.

Aspect 9: The bifunctional chelator compound according to any of the foregoing aspects, wherein the at least one radionuclide comprises ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, or ²²⁷Th.

Aspect 10: A bifunctional chelator compound comprising a formula (I)

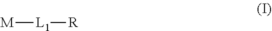

wherein R is a reactive group, wherein L₁ is a linker group, and wherein M is a chelator moiety.

Aspect 11: The bifunctional chelator of aspect 10, wherein L₁ comprises a formula (IA):

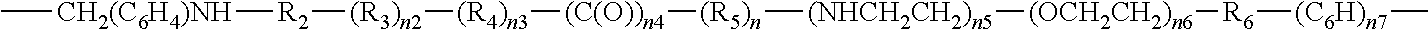

wherein n2-n7 is 0 or 1;
R₂ is —C(S)—, —C(S)NH—, —C(O)CH₂CH₂—, C(O)CH₂CH₂C(O)—;
R₃ is CH₂, NHCH₂, or (CH₂)ₙ₁, wherein n1 is 1 to 5;
R₄ is a cyclic alkane having 5 to 8 carbons or linear alkane having 1 to 6 carbons;
R₅ is an amino acid or amino acid derivative, wherein n is 1, 2, or 3; and
R₆ is —NHC(S)NH—, —C(S)NH, —C(O)NH—, NH(CH₂)₂NHC(O)(CH₂)₂, or NHC(O)(CH₂)₂—.

Aspect 12: The bifunctional chelator compound according to any of the foregoing aspects, further comprising at least one radionuclide, wherein the radionuclide is chelated by the bifunctional chelator.

Aspect 13: The bifunctional chelator compound according to any of the foregoing aspects, wherein the at least one radionuclide comprises ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, or ²²⁷Th.

Aspect 14: A bifunctional chelator compound comprising a formula (II)

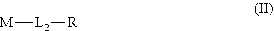

wherein R is a reactive group, wherein L₂ is a linker group, and wherein M is a chelator moiety.

Aspect 15: The bifunctional chelator of aspect 14, wherein L₂ may include the formula (IIA):

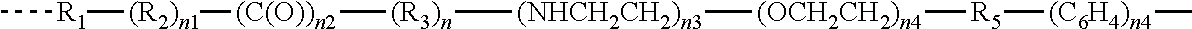

wherein n1-n5 is 0 or 1;
R₁ is —CH₂C(O)NHCH₂—, —C(CO₂H)CH₂CH₂C(O)NHCH₂—, —C(CO₂H)CH₂CH₂C(O)—, or —C(CO₂H)CH₂CH₂;
R₂ is a cyclic alkane having 5 to 8 carbons or linear alkane having 1 to 6 carbons;
R₃ is an amino acid or amino acid derivative, wherein n is 1, 2, or 3; and
R₄ is —NHC(S)NH—, —C(S)NH—, —NHC(O)CH₂CH₂C(O)NH—, —NHC(O)CH₂CH₂C(O)—, —NHC(O)CH₂CH₂—, —C(O)CH₂CH₂C(O)NH—, or —C(O)CH₂CH₂C(O)—.

Aspect 16: The bifunctional chelator compound according to any of the foregoing aspects, further comprising at least one radionuclide, wherein the radionuclide is chelated by the bifunctional chelator.

Aspect 17: The bifunctional chelator compound according to any of the foregoing aspects wherein the at least one radionuclide comprises ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, or ²²⁷Th.

Aspect 18: A bifunctional chelator compound comprising a formula (III):

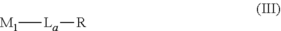

wherein R is a reactive group, wherein Lₐ is a linker group, and wherein M₁ is a chelator moiety.

Aspect 19: The bifunctional chelator compound according to aspect 18, wherein Lₐ may include the formula (IIIG):

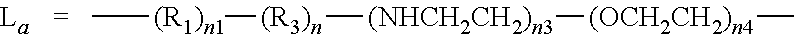

wherein n1-n4 is 0 or 1;
R₁ is —C(S)NHCH₂—(R₂)ₙ₂—C(O),
—C(S)NHNHCH₂—(R₂)ₙ₂—C(O), —NHCH₂—(R₂)ₙ₂—C(O),
—C(S)NH, —C(O)CH₂CH₂C(O)NH(R₂)C(O)—, —C(O)CH₂CH₂C(O)NHCH₂(R₂)C(O)—,
R₂C(O), or —C(O)CH₂CH₂C(O)NH—;
R₂ is a cyclohexane or CH₂R₂ is a cyclohexane or CH₂; and
R₃ is an amino acid or amino acid derivative, wherein n is 1, 2, or 3.

Aspect 20: The bifunctional chelator compound according to any of the foregoing aspects, wherein the chelator moiety is selected from:

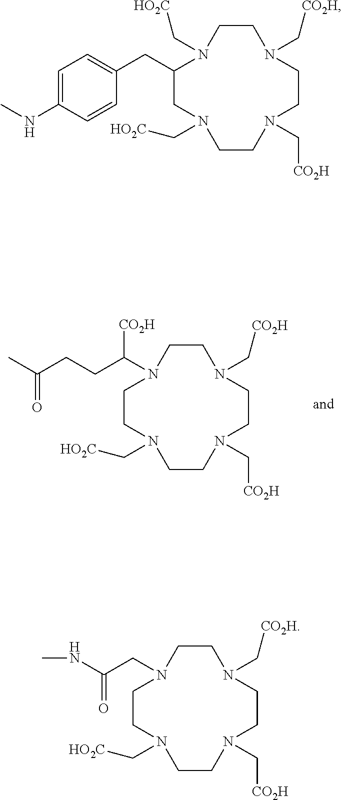 [SMILES: CNc1ccc(CC2CN(CC(=O)O)CCN(CC(=O)O)CCN(CC(=O)O)CCN2CC(=O)O)cc1 ; CC(=O)CCC(C(=O)O)N1CCN(CC(=O)O)CCN(CC(=O)O)CCN(CC(=O)O)CC1 ; CNC(=O)CN1CCN(CC(=O)O)CCN(CC(=O)O)CCN(CC(=O)O)CC1]

Aspect 21: The bifunctional chelator of claim according to any of the foregoing aspects, wherein the reactive group is selected from:

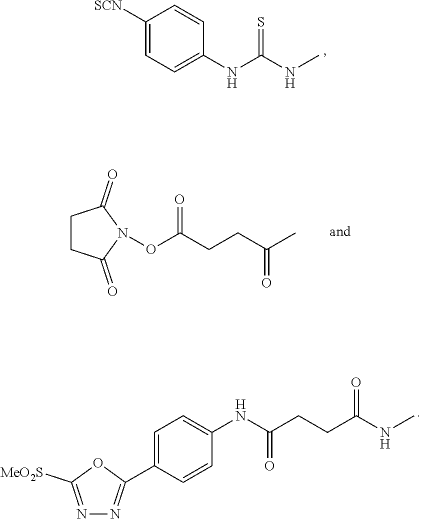

Aspect 22: The bifunctional chelator compound according to any of the foregoing aspects, further comprising at least one radionuclide, wherein the radionuclide is chelated by the bifunctional chelator.

Aspect 23: The bifunctional chelator compound according to any of the foregoing aspects, wherein the at least one radionuclide comprises ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, or ²²⁷Th.

Aspect 24: A conjugate of the bifunctional chelator compound of aspects 1-23 with a molecule comprising either of a primary amine group or a free thiol group.

Aspect 25: The conjugate of aspect 24, wherein the molecule comprises a peptide, a synthetic peptide, a protein, a recombinant protein, an antibody, a monoclonal antibody, an antigen-binding fragment of an antibody, an antigen-binding fragment of a monoclonal antibody, an immunoglobulin heavy chain, an immunoglobulin heavy chain variable region, immunoglobulin light chain, an immunoglobulin light chain variable region, a nanobody, a scFv molecule, or an antibody mimetic protein.

Aspect 26: The conjugate according to any of the foregoing aspects, wherein the molecule is a CD38 targeting agent.

Aspect 27: The conjugate according to any of the foregoing aspects, wherein the CD38 targeting agent is daratumumab, isatuximab, or MOR202.

Aspect 28: The conjugate according to any of the foregoing aspects, wherein the CD38 targeting agent is an antibody or an antibody fragment, comprising:
an immunoglobulin heavy chain having the amino acid sequence as set forth in SEQ ID NO:124 and/or an immunoglobulin light chain having an amino acid sequence as set forth in SEQ ID NO:128;
an immunoglobulin heavy chain having complementarity determining regions 1-3 with amino acid sequences as set forth in SEQ ID NOS: 125-127, respectively, and/or an immunoglobulin light chain having complementarity determining regions 1-3 with amino acid sequences as set forth in SEQ ID NOS: 129-131;
an immunoglobulin heavy chain having the amino acid sequence as set forth in SEQ ID NO:124 and/or an immunoglobulin light chain having complementarity determining regions 1-3 with amino acid sequences as set forth in SEQ ID NOS: 129-131; or
an immunoglobulin light chain having an amino acid sequence as set forth in SEQ ID NO:128, and/or an immunoglobulin heavy chain having complementarity determining regions 1-3 with amino acid sequences as set forth in SEQ ID NOS: 125-127, respectively.

Aspect 29: The conjugate according to any of the foregoing aspects, wherein the molecule is a TROP2 targeting agent, wherein the TROP2 targeting agent binds human TROP2.

Aspect 30: The conjugate according to any of the foregoing aspects, wherein the TROP2 targeting agent is an antibody, an antibody fragment, or an scFv molecule, comprising:
an immunoglobulin heavy chain having complementarity determining regions 1-3 of Sacituzumab and/or an immunoglobulin light chain having complementarity determining regions 1-3 of Sacituzumab;
an immunoglobulin heavy chain having complementarity determining regions 1-3 with amino acid sequences as set forth in SEQ ID NOS:68-70, respectively, and/or an immunoglobulin light chain having complementarity determining regions 1-3 with amino acid sequences as set forth in SEQ ID NOS:73-75, respectively;
an immunoglobulin heavy chain having complementarity determining regions 1-3 of Datopotamab and/or an immunoglobulin light chain having complementarity determining regions 1-3 of Datopotamab; or
an immunoglobin heavy chain having complementarity determining regions 1-3 with amino acid sequences as set forth in SEQ ID NOS: 78-80, respectively, and/or an immunoglobulin light chain having complementarity determining regions 1-3 with amino acid sequences as set forth in SEQ ID NOS: 83-85, respectively.

Aspect 31: The conjugate according to any of the foregoing aspects, comprising the immunoglobulin heavy chain having complementarity determining regions with amino acid sequences as set forth in SEQ ID NOS:68-70 and/or the immunoglobulin light chain having complementarity determining regions with amino acid sequences as set forth in SEQ ID NOS:73-75; or comprising the immunoglobulin heavy chain having complementarity determining regions with amino acid sequences as set forth in SEQ ID NOS: 78-80 and/or the immunoglobulin light chain having complementarity determining regions with amino acid sequences as set forth in SEQ ID NOS: 83-85.

Aspect 32: The conjugate according to any of the foregoing aspects, wherein the molecule is a 5T4 targeting agent, wherein the 5T4 targeting agent binds human 5T4.

Aspect 33: The conjugate according to any of the foregoing aspects, wherein the 5T4 targeting agent is naptumomab estafenatox.

Aspect 34: The conjugate according to any of the foregoing aspects, wherein the 5T4 targeting agent is an antibody, an antibody fragment or an scFv molecule, comprising an immunoglobulin heavy chain having complementarity determining regions 1-3 with amino acid sequences as set forth in SEQ ID NOS: 116-118, respectively, and/or immunoglobulin light chain having complementarity determining regions 1-3 with amino acid sequences as set forth in SEQ ID NOS:119-121, respectively.

Aspect 35: A composition comprising:
a quantity of a conjugate according to any of the foregoing aspects; and
at least one excipient.

Aspect 36: A composition comprising:
a quantity of a conjugate according to any of the foregoing aspects,
wherein the quantity of the conjugate comprises:
   a radiolabeled fraction, wherein the bifunctional chelator compound comprises a chelator moiety, wherein the chelator moiety chelates one or more radionuclides, and
   a non-radiolabeled fraction wherein the chelator moiety does not chelate a radionuclide.

Aspect 37: The composition according to any of the foregoing aspects, wherein the at least one radionuclide comprises one or more of ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, and ²²⁷Th.

Aspect 38: The composition according to any of the foregoing aspects, further comprising at least one excipient.

Aspect 39: A method for manufacturing a chelator-conjugated molecule, comprising the steps:

reacting a molecule comprising at least one primary amine group with a bifunctional chelator compound having a NHS or SCN reactive group according to any one of claims 1 through 23 to conjugate the bifunctional chelator compound to the molecule.

Aspect 40: The method of aspect 39, wherein the molecule comprises a peptide, a synthetic peptide, a protein, or a recombinant protein.

Aspect 41: The method according to any of the foregoing aspects, wherein the molecule comprises an antibody, a monoclonal antibody, an antigen-binding fragment of an antibody, an antigen-binding fragment of a monoclonal antibody, an immunoglobulin heavy chain, an immunoglobulin heavy chain variable region, an immunoglobulin light chain, an immunoglobulin light chain variable region, a nanobody, a scFv molecule, or an antibody mimetic protein.

Aspect 42: The method according to any of the foregoing aspects, wherein the molecule is a CD38 targeting agent.

Aspect 43: The method according to any of the foregoing aspects, wherein the CD38 targeting agent is daratumumab, isatuximab, or MOR202.

Aspect 44: The method according to any of the foregoing aspects, wherein the molecule comprises a peptide ligand of a receptor protein.

Aspect 45: The method according to any of the foregoing aspects, further comprising the step of chelating one or more radionuclides to the chelator moiety of the bifunctional chelator compound before and/or after performing the conjugation.

Aspect 46: The method according to any of the foregoing aspects, wherein the one or more radionuclide comprise one or more of ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, and ²²⁷Th.

Aspect 47: The method according to any of the foregoing aspects, wherein the molecule is a 5T4 targeting agent, wherein the 5T4 targeting agent binds human 5T4.

Aspect 48: The method according to any of the foregoing aspects, wherein the 5T4 targeting agent is naptumomab estafenatox.

Aspect 49: The method according to any of the foregoing aspects, wherein the 5T4 targeting agent is an antibody, an antibody fragment or an scFv molecule, comprising an immunoglobulin heavy chain having complementarity determining regions 1-3 with amino acid sequences as set forth in SEQ ID NOS:116-118, respectively, and/or immunoglobulin light chain having complementarity determining regions 1-3 with amino acid sequences as set forth in SEQ ID NOS: 119-121, respectively.

Aspect 50: The method according to any of the foregoing aspects, wherein the CD38 targeting agent is an antibody or an antibody fragment, comprising:
an immunoglobulin heavy chain having the amino acid sequence as set forth in SEQ ID NO:124 and/or an immunoglobulin light chain having an amino acid sequence as set forth in SEQ ID NO:128;
an immunoglobulin heavy chain having complementarity determining regions 1-3 with amino acid sequences as set forth in SEQ ID NOS: 125-127, respectively, and/or an immunoglobulin light chain having complementarity determining regions 1-3 with amino acid sequences as set forth in SEQ ID NOS: 129-131;
an immunoglobulin heavy chain having the amino acid sequence as set forth in SEQ ID NO:124 and/or an immunoglobulin light chain having complementarity determining regions 1-3 with amino acid sequences as set forth in SEQ ID NOS: 129-131; or
an immunoglobulin light chain having an amino acid sequence as set forth in SEQ ID NO:128, and/or an immunoglobulin heavy chain having complementarity determining regions 1-3 with amino acid sequences as set forth in SEQ ID NOS: 125-127, respectively.

Aspect 51: The method according to any of the foregoing aspects, wherein the CD38 targeting agent is an antibody or an antibody fragment, comprising any of the amino acid sequences as set forth in SEQ ID NOS: 124-139.

Aspect 52: A method for manufacturing a chelator-conjugated molecule, comprising the steps:
reacting a molecule comprising at least one free thiol group with a bifunctional chelator compound having a PODS reactive group according to any one of claims 7 through 23 to conjugate the bifunctional chelator compound to the molecule.

Aspect 53: The method of aspect 52, wherein the molecule comprises a peptide, a synthetic peptide, a protein, or a recombinant protein.

Aspect 54: The method according to any of the foregoing aspects, wherein the molecule comprises an antibody, a monoclonal antibody, an antigen-binding fragment of an antibody, an antigen-binding fragment of a monoclonal antibody, an immunoglobulin heavy chain, an immunoglobulin heavy chain variable region, an immunoglobulin light chain, an immunoglobulin light chain variable region, a nanobody, a scFv molecule, or an antibody mimetic protein.

Aspect 55: The method according to any of the foregoing aspects, wherein providing the molecule comprising at least one free thiol group further comprises reducing a disulfide bond to generate the at least one free thiol group of the molecule.

Aspect 56: The method according to any of the foregoing aspects, wherein the molecule comprises a peptide ligand of a receptor protein.

Aspect 57: The method according to any of the foregoing aspects, wherein the molecule is a CD38 targeting agent.

Aspect 58: The method according to any of the foregoing aspects, wherein the CD38 targeting agent is daratumumab.

Aspect 59: The method according to any of the foregoing aspects, wherein the CD38 targeting agent is an antibody or an antibody fragment, comprising:
an immunoglobulin heavy chain having the amino acid sequence as set forth in SEQ ID NO:124 and/or an immunoglobulin light chain having an amino acid sequence as set forth in SEQ ID NO:128;
an immunoglobulin heavy chain having complementarity determining regions 1-3 with amino acid sequences as set forth in SEQ ID NOS: 125-127, respectively, and/or an immunoglobulin light chain having complementarity determining regions 1-3 with amino acid sequences as set forth in SEQ ID NOS: 129-131;
an immunoglobulin heavy chain having the amino acid sequence as set forth in SEQ ID NO:124 and/or an immunoglobulin light chain having complementarity determining regions 1-3 with amino acid sequences as set forth in SEQ ID NOS: 129-131; or
an immunoglobulin light chain having an amino acid sequence as set forth in SEQ ID NO:128, and/or an immunoglobulin heavy chain having complementarity determining regions 1-3 with amino acid sequences as set forth in SEQ ID NOS: 125-127, respectively.

Aspect 60: The method according to any of the foregoing aspects, further comprising the step of chelating one or more radionuclides to the chelator moiety of the bifunctional chelator compound before and/or after performing the conjugation.

Aspect 61: The method according to any of the foregoing aspects, wherein the one or more radionuclide comprise one or more of ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, and ²²⁷Th.

Aspect 62: The method according to any of the foregoing aspects, wherein the molecule is a 5T4 targeting agent, wherein the 5T4 targeting agent binds human 5T4.

Aspect 63: The method according to any of the foregoing aspects, wherein the 5T4 targeting agent is naptumomab estafenatox.

Aspect 64: The method according to any of the foregoing aspects, wherein the 5T4 targeting agent is an antibody, an antibody fragment or an scFv molecule, comprising an immunoglobulin heavy chain having complementarity determining regions 1-3 with amino acid sequences as set forth in SEQ ID NOS:116-118, respectively, and/or an immunoglobulin light chain having complementarity determining regions 1-3 with amino acid sequences as set forth in SEQ ID NOS: 119-121, respectively.

Aspect 65: The method according to any of the foregoing aspects, wherein the CD38 targeting agent is an antibody or an antibody fragment, comprising any of the amino acid sequences as set forth in SEQ ID NOS: 124-139.

Aspect 66: A method for manufacturing a chelator-conjugated molecule, comprising the steps:
reacting a molecule comprising at least one primary amine group with a bifunctional chelator compound having a NHS or SCN reactive group according to any one of claims 1 through 23 to conjugate the bifunctional chelator compound to the molecule; and
reacting a molecule comprising at least one free thiol group with a bifunctional chelator compound having a PODS reactive group according to any one of claims 7 through 23 to conjugate the bifunctional chelator compound to the molecule.

Aspect 67: The method according to aspect 66, wherein the molecule comprises a peptide, a synthetic peptide, a protein, or a recombinant protein.

Aspect 68: The method according to any of the foregoing aspects, wherein the method comprises reacting the molecule comprising at least one free thiol group, the method further comprises reducing a disulfide bond to generate the at least one free thiol group of the molecule.

Aspect 69: The method according to any of the foregoing aspects, wherein the molecule comprises an antibody, a monoclonal antibody, an antigen-binding fragment of an antibody, an antigen-binding fragment of a monoclonal antibody, an immunoglobulin heavy chain, an immunoglobulin heavy chain variable region, an immunoglobulin light chain, an immunoglobulin light chain variable region, a nanobody, a scFv molecule, or an antibody mimetic protein.

Aspect 70: The method according to any of the foregoing aspects, wherein the molecule comprises a peptide ligand of a receptor protein.

Aspect 71: The method according to any of the foregoing aspects, wherein the molecule is a CD38 targeting agent.

Aspect 72: The method according to any of the foregoing aspects, wherein the CD38 targeting agent is daratumumab.

Aspect 73: The method according to any of the foregoing aspects, wherein the CD38 targeting agent is an antibody or an antibody fragment, comprising:
an immunoglobulin heavy chain having the amino acid sequence as set forth in SEQ ID NO:124 and/or an immunoglobulin light chain having an amino acid sequence as set forth in SEQ ID NO:128;
an immunoglobulin heavy chain having complementarity determining regions 1-3 with amino acid sequences as set forth in SEQ ID NOS: 125-127, respectively, and/or an immunoglobulin light chain having complementarity determining regions 1-3 with amino acid sequences as set forth in SEQ ID NOS: 129-131;
an immunoglobulin heavy chain having the amino acid sequence as set forth in SEQ ID NO:124 and/or an immunoglobulin light chain having complementarity determining regions 1-3 with amino acid sequences as set forth in SEQ ID NOS: 129-131; or
an immunoglobulin light chain having an amino acid sequence as set forth in SEQ ID NO:128, and/or an immunoglobulin heavy chain having complementarity determining regions 1-3 with amino acid sequences as set forth in SEQ ID NOS: 125-127, respectively.

Aspect 74: The method according to any of the foregoing aspects, further comprising the step of chelating one or more radionuclides to the chelator moiety of the bifunctional chelator compound before and/or after performing the conjugation.

Aspect 75: The method according to any of the foregoing aspects, wherein the one or more radionuclide comprise one or more of ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, and ²²⁷Th.

Aspect 76: The method according to any of the foregoing aspects, wherein the molecule is a 5T4 targeting agent, wherein the 5T4 targeting agent binds human 5T4.

Aspect 77: The method according to any of the foregoing aspects, wherein the 5T4 targeting agent is naptumomab estafenatox.

Aspect 78: The method according to any of the foregoing aspects, wherein the 5T4 targeting agent is an antibody, an antibody fragment or an scFv molecule, comprising an immunoglobulin heavy chain having complementarity determining regions 1-3 with amino acid sequences as set forth in SEQ ID NOS:116-118, respectively, and/or an immunoglobulin light chain having complementarity determining regions 1-3 with amino acid sequences as set forth in SEQ ID NOS: 119-121, respectively.

Aspect 79: The method according to any of the foregoing aspects, wherein the CD38 targeting agent is an antibody or an antibody fragment, comprising any of the amino acid sequences as set forth in SEQ ID NOS: 124-139.

Aspect 80: A chelator-conjugated molecule prepared according to the method of any one of aspects 39-79.

Aspect 81: The chelator-conjugated molecule of aspect 80, wherein the molecule is a 5T4 targeting agent that binds human 5T4.

Aspect 82: The chelator-conjugated molecule according to any of the foregoing aspects, wherein the 5T4 targeting agent is naptumomab estafenatox.

Aspect 83: The chelator-conjugated molecule according to any of the foregoing aspects, wherein the molecule is a CD38 targeting agent.

Aspect 84: The chelator-conjugated molecule according to any of the foregoing aspects, wherein the 5T4 targeting agent is an antibody, an antibody fragment or an scFv molecule, comprising an immunoglobulin heavy chain having complementarity determining regions 1-3 with amino acid sequences as set forth in SEQ ID NOS:116-118, respectively, and/or an immunoglobulin light chain having complementarity determining regions 1-3 with amino acid sequences as set forth in SEQ ID NOS: 119-121, respectively.

Aspect 85: A composition comprising:
a quantity of a chelator-conjugated molecule according to any of the foregoing aspects;
a quantity of a radionuclide;
wherein the quantity of the chelator-conjugated molecule comprises a radiolabeled fraction, wherein the chelator moiety chelates the radionuclide and a non-radiolabeled fraction, and wherein the chelator moiety does not chelate any radionuclide.

Aspect 86: The composition of aspect 85, further comprising at least one excipient.

Aspect 87: The composition according to any of the foregoing aspects, wherein the radionuclide is selected from ¹³⁴Ce, ⁴³Sc, ⁴⁴Sc, ⁴⁷Sc, ⁵⁵Co, ⁶⁰Cu, ⁶¹Cu, ⁶²Cu, ⁶Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, and ²²⁷Th.

Aspect 88: A method of manufacturing a chelator-conjugated molecule according to Example 1.

Aspect 89: A method of synthesizing a bifunctional chelator compound according to Example 2, as shown in FIGS. 18-21.

### EXAMPLES

### Example 1: Preparation of a Radiolabeled Antibody

Preparing a chelator-conjugated antibody using an SCN (thiocyanate) reactive group bifunctional chelator: antibody conjugates are prepared by reacting a concentrated solution of monoclonal antibody with a SCN reactive group bifunctional chelator as disclosed herein in bicarbonate or in phosphate buffers at pH between about 8 and about 9 and by incubation at cither about 37° C. or at room temperature. The molar ratio of antibody to bifunctional chelator is, for example, 1:10 or 1:20, or from 1:10 to 1:20. The conjugates are purified from excess of the bifunctional chelator by repeated filtration or centrifugation and by gravity size exclusion chromatography (SEC). During the purification process, the bicarbonate or phosphate buffer can be changed to N-2-Hydroxyethylpiperazine-N-2-ethanesulfonic acid (HEPES; Free Acid) or acetate medium. Conjugates are characterized by size exclusion high performance liquid chromatography (SE-HPLC).

Preparing a chelator-conjugated antibody using an NHS (NHS ester; N-hydroxysuccinimide ester) reactive group bifunctional chelator: The conjugation reaction between an antibody such as an IgG and an NHS ester reactive group bifunctional chelator is, for example, performed in 0.1 M NaHCO₃ buffer, pH 7.4, with 10- to 20-fold molar excess of bifunctional chelator to antibody, for 45 minutes to 1 hour, at room temperature/25° C. or 37° C. The conjugates are purified from excess of the bifunctional chelator by repeated filtration or centrifugation and by gravity size exclusion chromatography (SEC). During the purification process, the bicarbonate buffer can be changed to N-2-Hydroxyethylpiperazine-N-2-ethanesulfonic acid (HEPES; Free Acid) or acetate medium. Conjugates are characterized by size exclusion high performance liquid chromatography (SE-HPLC).

Preparing a chelator-conjugated antibody using a PODS (phenyloxadiazolyl methylsulfone) reactive group bifunctional chelator: A PODS reactive group bifunctional linker is conjugated to a monoclonal antibody, such as an IgG in the presence of TCEP, a mild reducing agent that reduces the inter-chain disulfide bonds within an immunoglobin to provide free thiols. The conjugation can be performed according to the methods set forth in U.S. Pat. No. 11,000,604.

In more detail, to a solution of 200 μg of antibody in PBS pH 7.4 (1 mg/mL) 1.33 μL of a fresh TCEP solution (10 mM in water, 10 eq.) is added concurrent with or followed by adding a suitable volume of a solution of a PODS reactive group bifunctional chelator (1 mM in DMSO). The reaction mixture is then stirred on a thermomixer (25° C. or 37° C.) for 30 min, 2 h, or 24 h. The resulting conjugate is then be purified on a size exclusion column (Sephadex G-25 M, PD-10 column, GE Healthcare; dead volume=2.5 mL, eluted with 2 mL of PBS, pH 7.4) and concentrated using centrifugal filtration units with a 50,000 Da molecular weight cut off (AMICON™ Ultra 4 Centrifugal Filtration Units, Millipore Corp., Billerica, MA, USA). The buffer in which the conjugate is dissolved can be changed to N-2-Hydroxyethylpiperazine-N-2-ethanesulfonic acid (HEPES; Free Acid) or acetate medium.

Radiolabeling the conjugate: A chelator-bearing conjugate is first prepared according to the aforementioned examples and then radiolabeled. Where the chelator moiety includes DOTA, DOTAGA or a DOTA derivative as exemplified herein, one radionuclide that is chelated is 225 Ac.

An exemplary labeling reaction for ²²⁵Ac is as follows: A reaction including 15 μl 0.15M NH₄Oac buffer, pH=6.5 and 2 μL (10 μg) chelator-conjugated antibody (5 mg/ml) is mixed in an Eppendorf reaction tube, and 4 μL ²²⁵Ac (10 μCi) in 0.05 M HCl subsequently added. The contents of the tube are mixed with a pipette tip and the reaction mixture incubated at 37° C. for 90 min with shaking at 100 rpm. At the end of the incubation period, 3 μL of a 1 mM DTPA solution is added to the reaction mixture and incubated at room temperature for 20 min to bind the unreacted 225 Ac into the 225Ac-DTPA complex. Instant thin layer chromatography with 10 cm silica gel strip and 10 mM EDTA/normal saline mobile phase is used to determine the radiochemical purity of 225 Ac-chelator-conjugated antibody through separating 225 Ac-labeled antibody (225 Ac-chelator-conjugated antibody) from free 225 Ac (225 Ac-DTPA). In this system, the radiolabeled antibody stays at the point of application and 225 Ac-DTPA moves with the solvent front. The strips are cut in halves and counted in the gamma counter equipped with the multichannel analyzer using channels 72-110 for 225 Ac to exclude its daughters.

Purification: An exemplary radiolabeled targeting agent, such as 225Ac-chelator-conjugated antibody, is purified either on PD10 columns pre-blocked with 1% I or on Vivaspin centrifugal concentrators with a 50 kDa MW cut-off with 2×1.5 mL washes, 3 min per spin. HPLC analyses of the ²²⁵Ac-chelator-conjugated antibody after purification is conducted using a Waters HPLC system equipped with flow-through Waters UV and Bioscan Radiation detectors, using a TSK3000SW XL column eluted with PBS at pH=7.4 and a flow rate of 1 ml/min.

With respect to any of the bifunctional chelator embodiments disclosed herein that include a cyclohexyl group adjacent to DOTA or a different chelator moiety, the invention also provides corresponding embodiments in which the cyclohexyl group is replaced by a benzyl group. Similarly, with respect to any of the bifunctional chelator embodiments disclosed herein that include a benzyl group adjacent to DOTA or a different chelator moiety, the invention also provides corresponding embodiments in which the benzyl group is replaced by a cyclohexyl group.

### Example 2: Solid Phase Synthesis of Bifunctional Chelator Compounds

Synthesis of the Bifunctional Chelator Compound A with PEG3

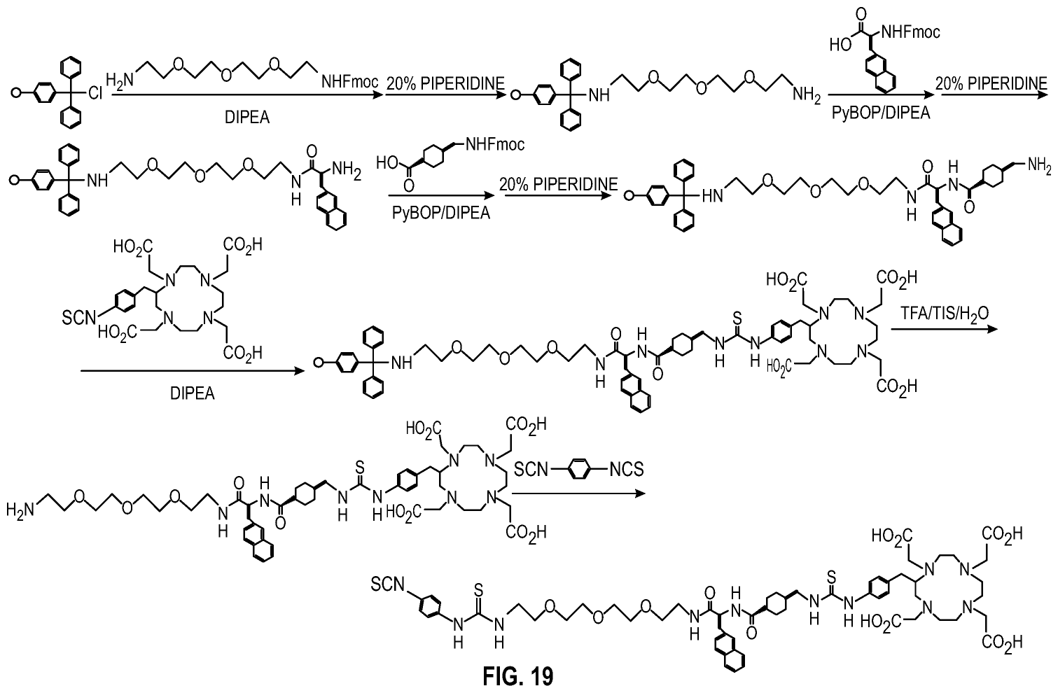

The synthesis was performed as shown in FIG. 19, wherein the starting materials and commercial sources thereof are described in Table 1.

TABLE 1

| Name | Structure | Supplier | Catalog No. |
|---|---|---|---|
| p-SCN-Bn-DOTA | 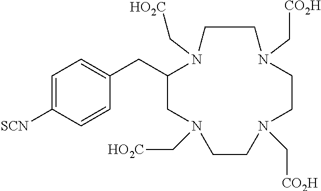 | Macrocyclics, Inc. (Plano, TX USA) | B-205 |
| Trityl chloride resin | 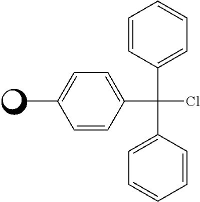 [SMILES: Cc1ccc(C(Cl)(c2ccccc2)c2ccccc2)cc1] | Chem-Impex Intl. Inc. (Wood Dale, IL USA) | 03902 |
| Fmoc-Amino-PEG3- Amine | 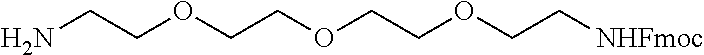 | Conju-Probe, LLC (San Diego, CA USA) | CP-1043 |
| Fmoc-3-(2-naphthyl)- alanine | 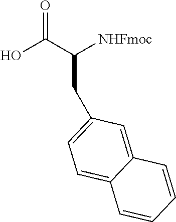 | Chem-Impex | 02588 |
| Fmoc-aminomethyl- cyclohexane carboxylic acid | 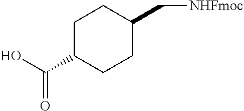 | Sigma- Aldrich (Burlington, MA USA; “Sigma”) | 58446 |
| p-phenylene diisothiocyanate | 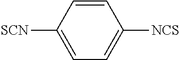 | Sigma | 258555 |

### Synthesis of the Bifunctional Chelator Compound A

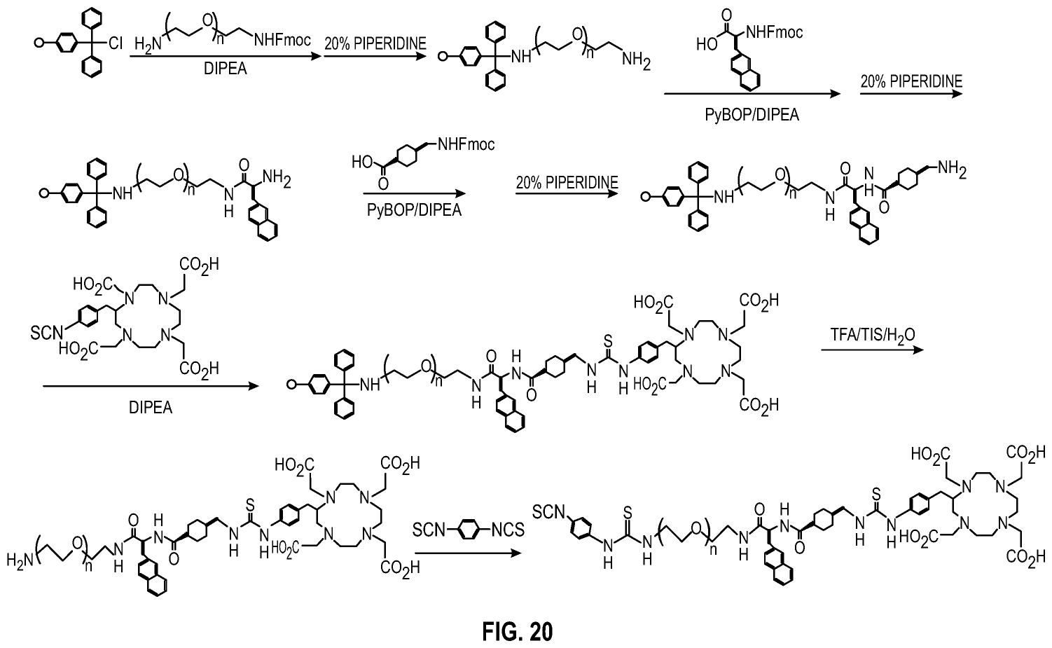

The synthesis was performed as shown in FIG. 20, wherein the starting materials are described in Table 2.

TABLE 2

| Name | Structure | Supplier | Catalog No. |
|---|---|---|---|
| p-SCN-Bn-DOTA | 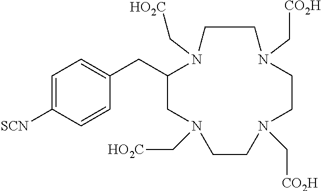 | Macrocyclics | B-205 |
| Trityl chloride resin | 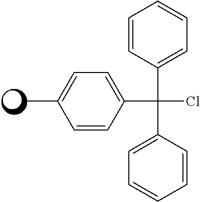 [SMILES: Cc1ccc(C(Cl)(c2ccccc2)c2ccccc2)cc1] | Chem-Impex | 03902 |
| Fmoc-NH-PEG-NH2, 10k | 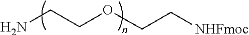 | BioPharma PEG Scientific Inc. (Watertown, MA USA) | HE069005-10K |
| Fmoc-3-(2-naphthyl)- alanine | 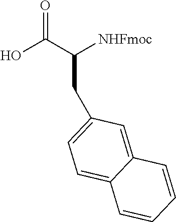 | Chem-Impex | 02588 |
| Fmoc-aminomethyl- cyclohexane carboxylic acid | 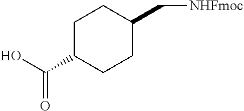 | Sigma | 58446 |
| p-phenylene diisothiocyanate | 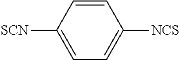 | Sigma | 258555 |

### Synthesis of the Bifunctional Chelator Compound B

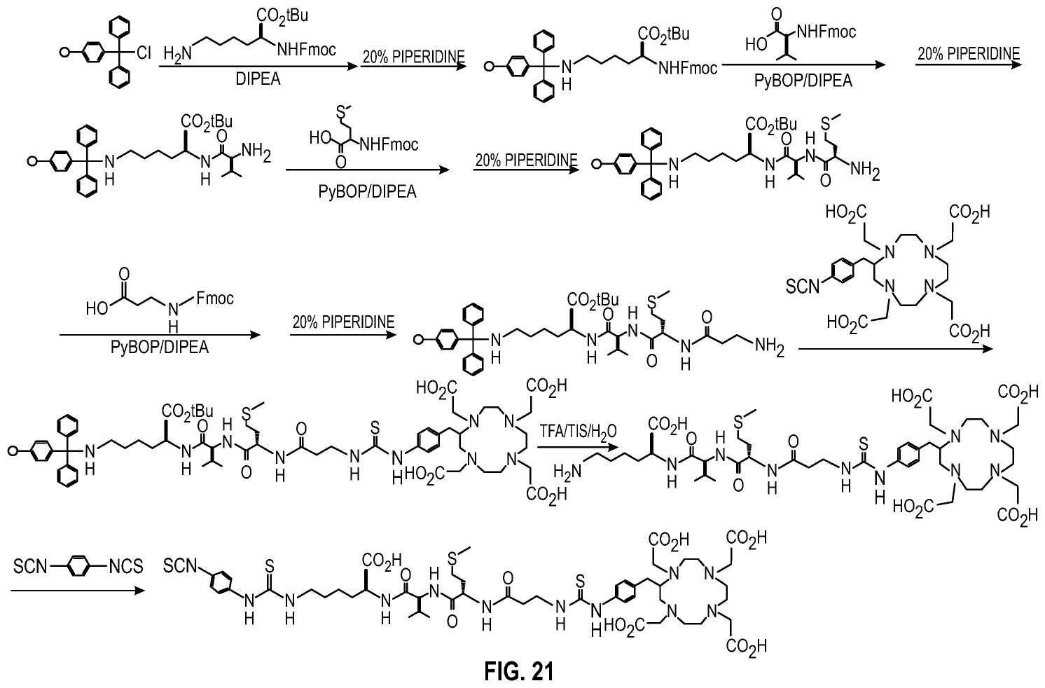

The synthesis was performed as shown in FIG. 21, wherein the starting materials are described in Table 3.

TABLE 3

| Name | Structure | Supplier | Catalog No. |
|---|---|---|---|
| Trityl chloride resin | 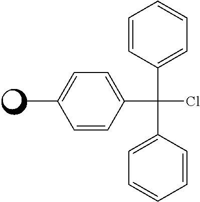 [SMILES: Cc1ccc(C(Cl)(c2ccccc2)c2ccccc2)cc1] | Chem-Impex | 03902 |
| p-SCN-Bn-DOTA | 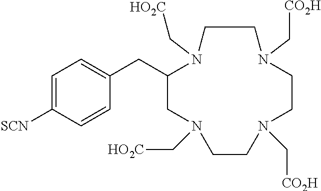 | Macrocyclics | B-205 |
| Fmoc-Lys-OtBu | 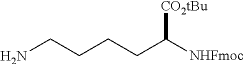 | Chem-Impex | 33496 |
| Fmoc-Val-OH | 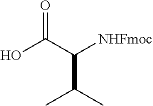 | Sigma | 47638 |
| Fmoc-Met-OH | 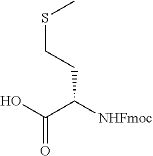 | Sigma | 47634 |
| Fmoc-beta-Ala-OH | 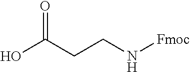 | Sigma | 47587 |
| p-phenylene diisothiocyanate | 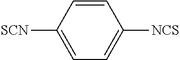 | Sigma | 258555 |

### Synthesis of the Bifunctional Chelator Compound C

The synthesis was performed as shown in FIG. 18.

1-9. (canceled)

10. A bifunctional chelator compound comprising a formula 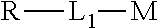
wherein R is a reactive group;
wherein L₁ comprises a formula 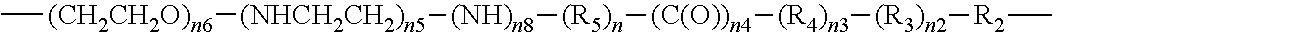
wherein:
R₂ is —C(S)—, —NHC(S)—, —CH₂CH₂C(O)—, and C(O)CH₂CH₂C(O)—;
R₃ is CH₂, CH₂NH, or (CH₂)ₙ₁, where n1 is an integer from 1 to 5;
R₄ is a cyclic alkane having 5 to 8 carbons or linear alkane having 1 to 6 carbons; and
R₅ is an amino acid or amino acid derivative;
wherein n is an integer selected from the group consisting of 1, 2, and 3; and
wherein each of n2, n3, n4, n5, no, n7, and ng is independently 0 or 1; and
wherein M is a chelator moiety.

11. The bifunctional chelator compound of claim 10, comprising a structure selected from a group consisting of:

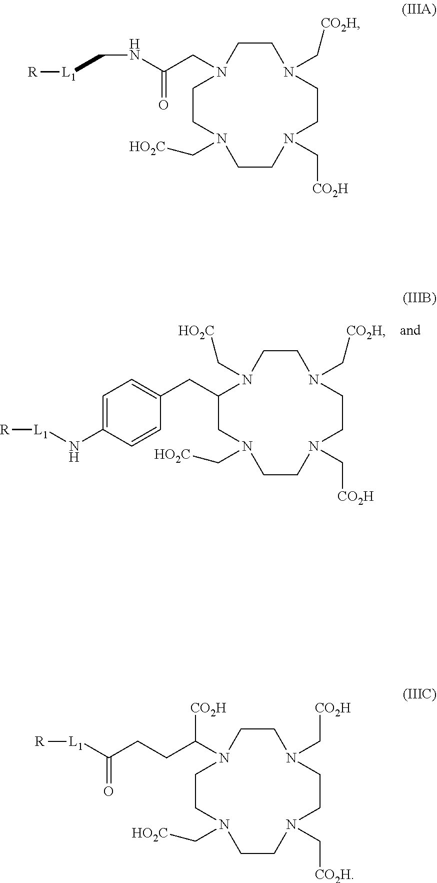

12. The bifunctional chelator compound of claim 10, comprising a structure selected from a group consisting of:

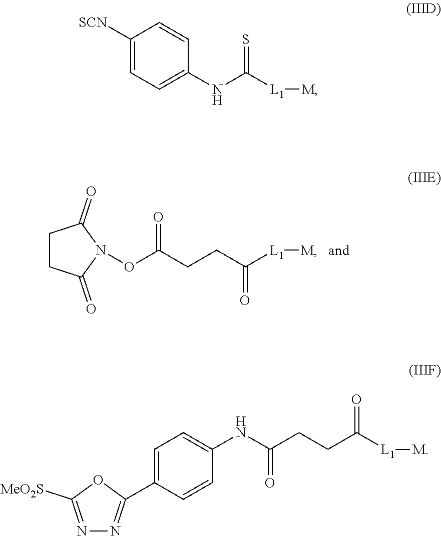

13. The bifunctional chelator of claim 10, wherein R₅ is -methionine-valine-lysine- or -lysine-valine-methionine-.

14. The bifunctional chelator of claim 10, comprising a structure

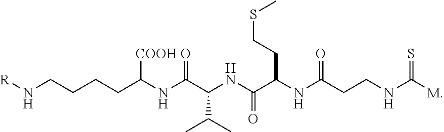

15. The bifunctional chelator of claim 10, comprising a structure

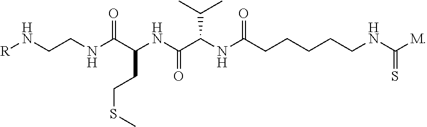

16. The bifunctional chelator of claim 10, comprising a structure

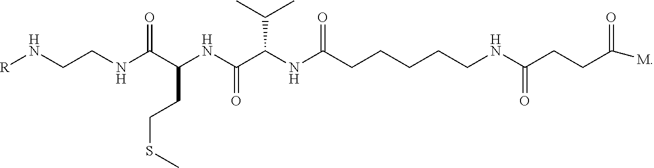

17. The bifunctional chelator of claim 10, comprising a structure

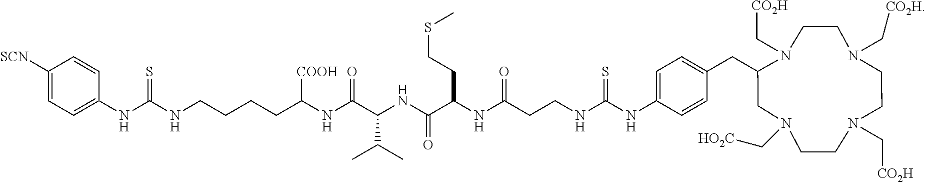

18. The bifunctional chelator of claim 10, comprising a structure

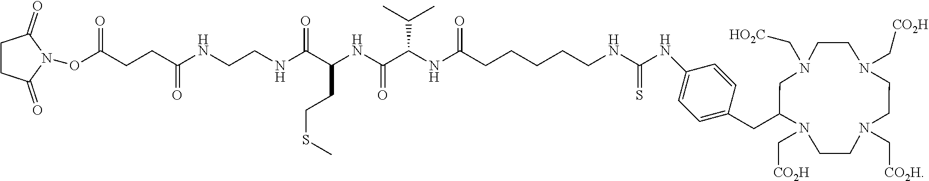 [SMILES: CSCC[C@H](NC(=O)[C@@H](NC(=O)CCCCCNC(=S)Nc1ccc(CC2CN(CC(=O)O)CCN(CC(=O)O)CCN(CC(=O)O)CCN2CC(=O)O)cc1)C(C)C)C(=O)NCCNC(=O)CCC(=O)ON1C(=O)CCC1=O]

19. The bifunctional chelator of claim 10, comprising a structure

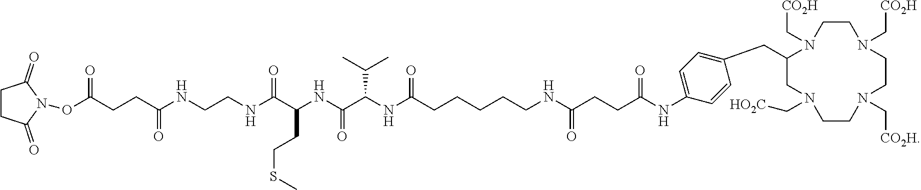 [SMILES: CSCC[C@H](NC(=O)[C@@H](NC(=O)CCCCCNC(=O)CCC(=O)Nc1ccc(CC2CN(CC(=O)O)CCN(CC(=O)O)CCN(CC(=O)O)CCN2CC(=O)O)cc1)C(C)C)C(=O)NCCNC(=O)CCC(=O)ON1C(=O)CCC1=O]

20. The bifunctional chelator compound of claim 11, comprising the structure selected from a group consisting of:

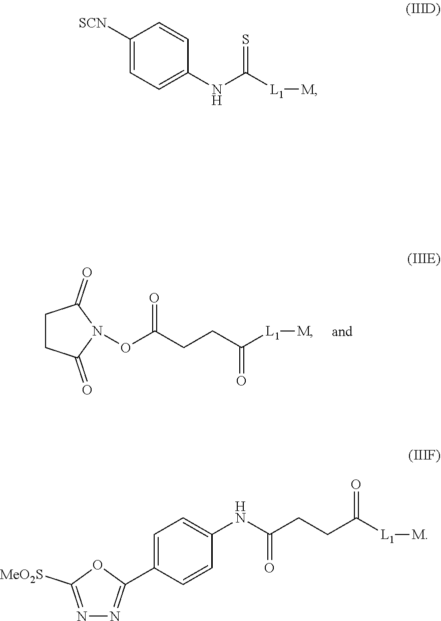

21. The bifunctional chelator of claim 10, comprising the structure selected from the group consisting of:

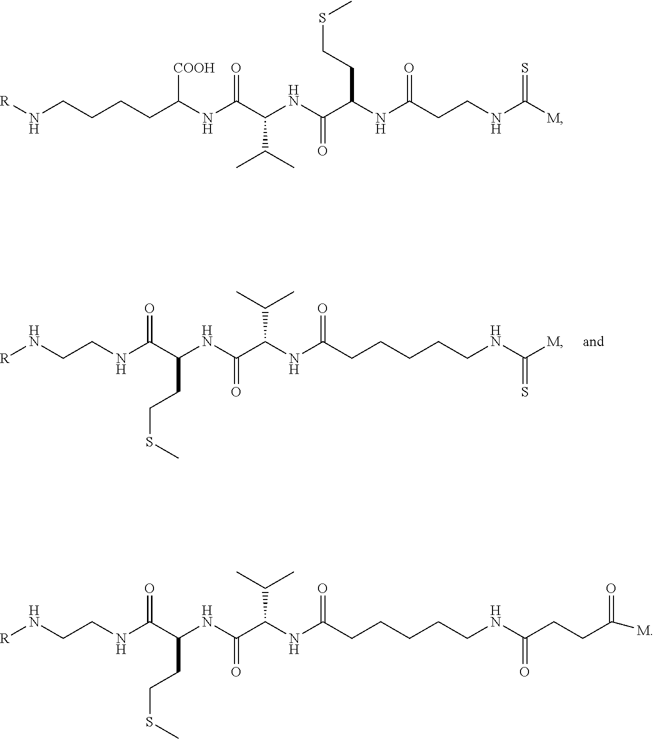

22. The bifunctional chelator of claim 10, further comprising at least one radionuclide, wherein the at least one radionuclide is chelated by the chelator moiety, and wherein the at least one radionuclide comprises ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ¹⁶⁶Ho, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Th, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²¹¹At, or ²²⁷Th.

23. A conjugate of the bifunctional chelator compound of claim 10 with a molecule comprising a primary amine group or a free thiol group.

24. The conjugate of claim 23, wherein the molecule comprising a primary amine group or a free thiol group is a peptide or a protein.

25. The conjugate of claim 23, wherein the molecule comprising a primary amine group or a free thiol group comprises an antibody, a monoclonal antibody, an antigen-binding fragment of an antibody, an antigen-binding fragment of a monoclonal antibody, an immunoglobulin heavy chain, an immunoglobulin heavy chain variable region, immunoglobulin light chain, an immunoglobulin light chain variable region, a nanobody, a scFv molecule, or an antibody mimetic protein.

26. A method for manufacturing a chelator-conjugated molecule, comprising:
reacting a molecule comprising at least one primary amine group with a bifunctional chelator compound according to claim 10 wherein the reactive group is a N-hydroxysuccinimide ester (NHS) reactive group or an isothiocyanate (SCN) reactive group, to conjugate the bifunctional chelator compound to the molecule; or
reacting a molecule comprising at least one free thiol group with a bifunctional chelator compound according to claim 1 wherein the reactive group is a phenyloxadiazolyl methylsulfone (PODS) reactive group, to conjugate the bifunctional chelator compound to the molecule.

27. A method for manufacturing a chelator-conjugated molecule, comprising: reacting a molecule comprising at least one free thiol group with a bifunctional chelator compound according to claim 10, wherein the reactive group is a phenyloxadiazolyl methylsulfone (PODS) reactive group, to conjugate the bifunctional chelator compound to the molecule; and reducing a disulfide bond to generate the at least one free thiol group of the molecule.

28. The method of claim 26, wherein the molecule comprising at least one primary amine group is a peptide or a protein, and the molecule comprising at least one free thiol group is a peptide or a protein.

29. The method of claim 26, wherein the molecule comprising at least one primary amine group is an antibody, a monoclonal antibody, an antigen-binding fragment of an antibody, an antigen-binding fragment of a monoclonal antibody, an immunoglobulin heavy chain, an immunoglobulin heavy chain variable region, an immunoglobulin light chain, an immunoglobulin light chain variable region, a nanobody, a scFv molecule, or an antibody mimetic protein; and
the molecule comprising at least one free thiol group is an antibody, a monoclonal antibody, an antigen-binding fragment of an antibody, an antigen-binding fragment of a monoclonal antibody, an immunoglobulin heavy chain, an immunoglobulin heavy chain variable region, an immunoglobulin light chain, an immunoglobulin light chain variable region, a nanobody, a scFv molecule, or an antibody mimetic protein.

30. The method of claim 26, further comprising chelating a radionuclide to the chelator moiety of the bifunctional chelator compound before or after performing the conjugation.


SEQUENCE LISTING (139 sequences)

SEQ ID NO: 1 - protein, 11 aa, synthetic construct
  REGION 1..11: note = Antibody component sequence
SASQDISNYLN

SEQ ID NO: 2 - protein, 7 aa, synthetic construct
  REGION 1..7: note = Antibody component sequence
DTSILHL

SEQ ID NO: 3 - protein, 9 aa, synthetic construct
  REGION 1..9: note = Antibody component sequence
QQYSKFPRT

SEQ ID NO: 4 - protein, 5 aa, synthetic construct
  REGION 1..5: note = Antibody component sequence
NYAMN

SEQ ID NO: 5 - protein, 17 aa, synthetic construct
  REGION 1..17: note = Antibody component sequence
WINTHTGDPTYADDFKG

SEQ ID NO: 6 - protein, 9 aa, synthetic construct
  REGION 1..9: note = Antibody component sequence
TYGNYAMDY

SEQ ID NO: 7 - protein, 107 aa, synthetic construct
  REGION 1..107: note = Antibody component sequence
DIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIK

SEQ ID NO: 8 - protein, 191 aa, synthetic construct
  REGION 1..191: note = Antibody component sequence
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSAKTTAPSVYPLAPVCGDTTGSSVTLGCLVKGYFPEPVTLTWNSGSLSSGVHTFPAVLQSDLYTLSSSVTVTSS

SEQ ID NO: 9 - protein, 109 aa, synthetic construct
  REGION 1..109: note = Antibody component sequence
DIQLTQSPSFLSASVGDRVTITCRASQGITSYLAWYQQKPGKAPKLLIYAASALQSGVPSRFSGRGSGTEFTLTISSLQPEDFATYYCQQVNRGAAITFGHGTRLDIKR

SEQ ID NO: 10 - protein, 120 aa, synthetic construct
  REGION 1..120: note = Antibody component sequence
  VARIANT 20: note = misc_feature - Xaa can be any naturally occurring amino acid
  VARIANT 35: note = misc_feature - Xaa can be any naturally occurring amino acid
QVQLVQSGAEVKKPGSSVRXSCRASGGSSTTYAFXWVRQAPGQGLEWMGGIVPIFGTLKYAQKFQDRVTLTADKSTGTAYMELNSLRLDDTAVYYCARAIQLEGRPFDHWGQGTQVTVSA

SEQ ID NO: 11 - protein, 11 aa, synthetic construct
  REGION 1..11: note = Antibody component sequence
QASQDIGNNLI

SEQ ID NO: 12 - protein, 7 aa, synthetic construct
  REGION 1..7: note = Antibody component sequence
YATNLAN

SEQ ID NO: 13 - protein, 7 aa, synthetic construct
  REGION 1..7: note = Antibody component sequence
QQWSSNP

SEQ ID NO: 14 - protein, 5 aa, synthetic construct
  REGION 1..5: note = Antibody component sequence
NYYMS

SEQ ID NO: 15 - protein, 17 aa, synthetic construct
  REGION 1..17: note = Antibody component sequence
NIYGGNGGTGYNQKFKG

SEQ ID NO: 16 - protein, 8 aa, synthetic construct
  REGION 1..8: note = Antibody component sequence
GDLYAMDY

SEQ ID NO: 17 - protein, 98 aa, synthetic construct
  REGION 1..98: note = Antibody component sequence
VLTQSPSSMSASLGDRVTITCQASQDIGNNLIWFQQKPGKSPRPMIYYATNLANGVPSRFSGSGSGTSYSLTISSMEAEDAATYYCQQWSSNPYTFGG

SEQ ID NO: 18 - protein, 106 aa, synthetic construct
  REGION 1..106: note = Antibody component sequence
AELVKPGASVKLSCKTSGYTFSNYYMSWLKQMPGQNIEWIGNIYGGNGGTGYNQKFKGKATLTVDKSSSTAYMQLSSLTSEDSAVYFCARGDLYAMDYWGQGTTVT

SEQ ID NO: 19 - protein, 245 aa, synthetic construct
  REGION 1..245: note = Antibody component sequence
MAQVQLQQSGAELVKPGASVKLSCKTSGYTFSNYYMSWLKQMPGQNIEWIGNIYGGNGGTGYNQKFKGKATLTVDKSSSTAYMQLSSLTSEDSAVYFCARGDLYAMDYWGQGTTVTVSSGGGGSGGGGSGGGGSDIVLTQSPSSMSASLGDRVTITCQASQDIGNNLIWFQQKPGKSPRPMIYYATNLANGVPSRFSGSGSGTSYSLTISSMEAEDAATYYCQQWSSNPYTFGGGTKLEIKRAAA

SEQ ID NO: 20 - protein, 11 aa, synthetic construct
  REGION 1..11: note = Antibody component sequence
RASQNIDTSIH

SEQ ID NO: 21 - protein, 7 aa, synthetic construct
  REGION 1..7: note = Antibody component sequence
YASESIS

SEQ ID NO: 22 - protein, 9 aa, synthetic construct
  REGION 1..9: note = Antibody component sequence
QQSNYWPLT

SEQ ID NO: 23 - protein, 5 aa, synthetic construct
  REGION 1..5: note = Antibody component sequence
SYWMN

SEQ ID NO: 24 - protein, 6 aa, synthetic construct
  REGION 1..6: note = Antibody component sequence
GYSFTS

SEQ ID NO: 25 - protein, 10 aa, synthetic construct
  REGION 1..10: note = Antibody component sequence
GYSFTSYWMN

SEQ ID NO: 26 - protein, 17 aa, synthetic construct
  REGION 1..17: note = Antibody component sequence
MIHPSDSETRLNQKFKD

SEQ ID NO: 27 - protein, 10 aa, synthetic construct
  REGION 1..10: note = Antibody component sequence
MIHPSDSETR

SEQ ID NO: 28 - protein, 9 aa, synthetic construct
  REGION 1..9: note = Antibody component sequence
EMGPYTLDY

SEQ ID NO: 29 - protein, 127 aa, synthetic construct
  REGION 1..127: note = Antibody component sequence
MSVPTQVLGLLLLWLTDARCDILLTQSPAILSVSPGARVSFSCRASQNIDTSIHWYQQRTNGSPRLLIKYASESISGIPSRFSGSGSGTDFTLSINSVESEDIADYYCQQSNYWPLTFGAGTKLELK

SEQ ID NO: 30 - protein, 141 aa, synthetic construct
  REGION 1..141: note = Antibody component sequence
MEWSWVFLFFLSVTTGVHSQVQLQQPGAELVRPGASVKLSCKASGYSFTSYWMNWMKQRPGQGLEWIGMIHPSDSETRLNQKFKDKATLTVDKSSSTAYMQLNSPTSEDSAVYYCAREMGPYTLDYWGQGTSVTVSSASTK

SEQ ID NO: 31 - protein, 16 aa, synthetic construct
  REGION 1..16: note = Antibody component sequence
KSSQSLLNSSNQKNYL

SEQ ID NO: 32 - protein, 7 aa, synthetic construct
  REGION 1..7: note = Antibody component sequence
FASTRES

SEQ ID NO: 33 - protein, 9 aa, synthetic construct
  REGION 1..9: note = Antibody component sequence
QQHYSTPPT

SEQ ID NO: 34 - protein, 5 aa, synthetic construct
  REGION 1..5: note = Antibody component sequence
RYAMS

SEQ ID NO: 35 - protein, 6 aa, synthetic construct
  REGION 1..6: note = Antibody component sequence
GFTFSR

SEQ ID NO: 36 - protein, 10 aa, synthetic construct
  REGION 1..10: note = Antibody component sequence
GFTFSRYAMS

SEQ ID NO: 37 - protein, 15 aa, synthetic construct
  REGION 1..15: note = Antibody component sequence
TIFSGGSYTYYPDSV

SEQ ID NO: 38 - protein, 8 aa, synthetic construct
  REGION 1..8: note = Antibody component sequence
TIFSGGSY

SEQ ID NO: 39 - protein, 9 aa, synthetic construct
  REGION 1..9: note = Antibody component sequence
PNWERTFDY

SEQ ID NO: 40 - protein, 133 aa, synthetic construct
  REGION 1..133: note = Antibody component sequence
MESQTQVLMFLLLWVSGACTDIVMTQSPSSLAMSVGQKVTMSCKSSQSLLNSSNQKNYLAWYQQKPGQSPKLLVYFASTRESGVPDRFMGSGSGTDFTLTISSVQAEDLADYFCQQHYSTPPTFGGGTKLEIK

SEQ ID NO: 41 - protein, 133 aa, synthetic construct
  REGION 1..133: note = Antibody component sequence
MRVLILLWLFTAFPGLLSDVQLQESGPGLVKPSQSLSLTCTVTGYSITSDYAWNWIRQFPGNKLEWMGFVSYSGTTKYNPSLKSRISITRDTSENQFFLQLNSVTSEDTATYYCARGYGFDYWGQGTTLTVSS

SEQ ID NO: 42 - protein, 38 aa, synthetic construct
  REGION 1..38: note = Antibody component sequence
EIILTQSPTTMAASPGEKITITCSASSSISSHYLHWYQ

SEQ ID NO: 43 - protein, 40 aa, synthetic construct
  REGION 1..40: note = Antibody component sequence
QKSGFSPKLLYRTSNLASGVPARESGSGSGTSYSLTIGTM

SEQ ID NO: 44 - protein, 29 aa, synthetic construct
  REGION 1..29: note = Antibody component sequence
EAEDVATYYCQQGSSLPLTFGAGTKVEIK

SEQ ID NO: 45 - protein, 41 aa, synthetic construct
  REGION 1..41: note = Antibody component sequence
EIQLQQSGPELVKPGASVKVSCKASGYAFTSQNIYWVKQSH

SEQ ID NO: 46 - protein, 40 aa, synthetic construct
  REGION 1..40: note = Antibody component sequence
GKSLEWIGYEPYNVVPMYNPKFKGKATLTVDKSSSSAYIH

SEQ ID NO: 47 - protein, 35 aa, synthetic construct
  REGION 1..35: note = Antibody component sequence
LNSLTSEDSAIYYCARSGSSNFDYWGQGTTLTVSS

SEQ ID NO: 48 - protein, 330 aa, Homo sapiens [sequence omitted]

SEQ ID NO: 49 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 50 - protein, 4 aa, synthetic construct
  REGION 1..4: note = fusion protein linker sequence
IEGR

SEQ ID NO: 51 - skipped

SEQ ID NO: 52 - protein, 345 aa, synthetic construct
  REGION 1..345: note = Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 345 aa, synthetic construct; identical to SEQ ID NO: 52
  REGION 1..345: note = Fusion protein

SEQ ID NO: 54 - protein, 347 aa, synthetic construct
  REGION 1..347: note = Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - DNA, 25 bp, synthetic construct
  misc_feature 1..25: note = Morpholino
  source 1..25: mol_type = other DNA
cgtcacaggcaggacccactgccca

SEQ ID NO: 56 - protein, 5 aa, synthetic construct
  REGION 1..5: note = Description of Artificial Sequence: Synthetic peptide
DYAMS

SEQ ID NO: 57 - protein, 17 aa, synthetic construct
  REGION 1..17: note = Description of Artificial Sequence: Synthetic peptide
TISDGGTYTYYPDSVKG

SEQ ID NO: 58 - protein, 10 aa, synthetic construct
  REGION 1..10: note = Description of Artificial Sequence: Synthetic peptide
EWGDYDGFDY

SEQ ID NO: 59 - protein, 11 aa, synthetic construct
  REGION 1..11: note = Description of Artificial Sequence: Synthetic peptide
RASQEISGYLS

SEQ ID NO: 60 - protein, 7 aa, synthetic construct
  REGION 1..7: note = Description of Artificial Sequence: Synthetic peptide
AASTLDS

SEQ ID NO: 61 - protein, 9 aa, synthetic construct
  REGION 1..9: note = Description of Artificial Sequence: Synthetic peptide
LQYDSYPYT

SEQ ID NO: 62 - protein, 119 aa, synthetic construct
  REGION 1..119: note = Description of Artificial Sequence: Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSS

SEQ ID NO: 63 - protein, 107 aa, synthetic construct
  REGION 1..107: note = Description of Artificial Sequence: Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIK

SEQ ID NO: 64 - protein, 471 aa, synthetic construct
  REGION 1..471: note = Description of Artificial Sequence: Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 236 aa, synthetic construct
  REGION 1..236: note = Description of Artificial Sequence: Synthetic polypeptide
MDMRVPAQLLGLLLLWLRGARCDIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 451 aa, synthetic construct
  REGION 1..451: note = mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 121 aa, synthetic construct
  REGION 1..121: note = mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 68 - protein, 5 aa, synthetic construct
  REGION 1..5: note = mAb
NYGMN

SEQ ID NO: 69 - protein, 17 aa, synthetic construct
  REGION 1..17: note = mAb
WINTYTGEPTYTDDFKG

SEQ ID NO: 70 - protein, 12 aa, synthetic construct
  REGION 1..12: note = mAb
GGFGSSYWYFDV

SEQ ID NO: 71 - protein, 214 aa, synthetic construct
  REGION 1..214: note = mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 110 aa, synthetic construct
  REGION 1..110: note = mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTV

SEQ ID NO: 73 - protein, 11 aa, synthetic construct
  REGION 1..11: note = mAb
KASQDVSIAVA

SEQ ID NO: 74 - protein, 7 aa, synthetic construct
  REGION 1..7: note = mAb
SASYRYT

SEQ ID NO: 75 - protein, 9 aa, synthetic construct
  REGION 1..9: note = mAb
QQHYITPLT

SEQ ID NO: 76 - protein, 451 aa, synthetic construct
  REGION 1..451: note = mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 121 aa, synthetic construct
  REGION 1..121: note = mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 78 - protein, 5 aa, synthetic construct
  REGION 1..5: note = mAb
TAGMQ

SEQ ID NO: 79 - protein, 17 aa, synthetic construct
  REGION 1..17: note = mAb
WINTHSGVPKYAEDFKG

SEQ ID NO: 80 - protein, 12 aa, synthetic construct
  REGION 1..12: note = mAb
SGFGSSYWYFDV

SEQ ID NO: 81 - protein, 214 aa, synthetic construct
  REGION 1..214: note = mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 110 aa, synthetic construct
  REGION 1..110: note = mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTV

SEQ ID NO: 83 - protein, 11 aa, synthetic construct
  REGION 1..11: note = mAb
KASQDVSTAVA

SEQ ID NO: 84 - protein, 7 aa, synthetic construct
  REGION 1..7: note = mAb
SASYRYT

SEQ ID NO: 85 - protein, 9 aa, synthetic construct
  REGION 1..9: note = mAb
QQHYITPLT

SEQ ID NO: 86 - protein, 450 aa, synthetic construct
  REGION 1..450: note = Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 214 aa, synthetic construct
  REGION 1..214: note = Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 448 aa, synthetic construct
  REGION 1..448: note = Antibody heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 214 aa, synthetic construct
  REGION 1..214: note = Antibody light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 244 aa, Homo sapiens
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 91 - protein, 233 aa, synthetic construct
  REGION 1..233: note = Mutated Protein
SEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 92 - protein, 233 aa, synthetic construct
  REGION 1..233: note = Mutated Protein
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTS

SEQ ID NO: 93 - protein, 679 aa, synthetic construct
  REGION 1..679: note = Conjugated Protein
  REGION 460..679: note = MISC_FEATURE - Light Chain
QVQLQQPGAELVRPGASVKLSCKASGYTFTNYWINWVKQRPGQGLEWIGNIYPSYIYTNYNQEFKDKVTLTVDESSSTAYMQLSSPTSEDSAVYYCTRSPYGYDEYGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTSDIVMTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 94 - protein, 672 aa, synthetic construct
  REGION 1..672: note = Mutated and Conjugated Protein
  REGION 459..672: note = MISC_FEATURE - Light Chain
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTSSIVMTQTPTSLLVSAGDRVTITCKASQSVSNDVAWYQQKPGQSPKLLISYTSSRYAGVPDRFSGSGSGTDFTLTISSVQAEDLAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 95 - protein, 672 aa, synthetic construct
  REGION 1..672: note = Mutated and Conjugated Protein
  REGION 459..672: note = MISC_FEATURE - Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 96 - protein, 458 aa, synthetic construct
  REGION 1..458: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 97 - protein, 214 aa, synthetic construct
  REGION 1..214: note = Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 98 - protein, 233 aa, synthetic construct
  REGION 1..233: note = Mutated Protein
SEKSEEINEKDLRKKSELQGTALGNLKQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIALYLYTT

SEQ ID NO: 99 - protein, 215 aa, synthetic construct
  REGION 1..215: note = Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESK

SEQ ID NO: 100 - protein, 215 aa, synthetic construct
  REGION 1..215: note = Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESC

SEQ ID NO: 101 - protein, 216 aa, synthetic construct
  REGION 1..216: note = Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESKC

SEQ ID NO: 102 - protein, 222 aa, synthetic construct
  REGION 1..222: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDS

SEQ ID NO: 103 - protein, 225 aa, synthetic construct
  REGION 1..225: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGP

SEQ ID NO: 104 - protein, 223 aa, synthetic construct
  REGION 1..223: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSK

SEQ ID NO: 105 - protein, 226 aa, synthetic construct
  REGION 1..226: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPK

SEQ ID NO: 106 - protein, 223 aa, synthetic construct
  REGION 1..223: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSC

SEQ ID NO: 107 - protein, 226 aa, synthetic construct
  REGION 1..226: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPC

SEQ ID NO: 108 - protein, 224 aa, synthetic construct
  REGION 1..224: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSKC

SEQ ID NO: 109 - protein, 227 aa, synthetic construct
  REGION 1..227: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPKC

SEQ ID NO: 110 - protein, 673 aa, synthetic construct
  REGION 1..673: note = Mutated and Conjugated Protein
  REGION 459..672: note = MISC_FEATURE - Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESK

SEQ ID NO: 111 - protein, 459 aa, synthetic construct
  REGION 1..459: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTK

SEQ ID NO: 112 - protein, 673 aa, synthetic construct
  REGION 1..673: note = Mutated and Conjugated Protein
  REGION 459..672: note = MISC_FEATURE - Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESC

SEQ ID NO: 113 - protein, 459 aa, synthetic construct
  REGION 1..459: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTC

SEQ ID NO: 114 - protein, 674 aa, synthetic construct
  REGION 1..674: note = Mutated and Conjugated Protein
  REGION 459..672: note = MISC_FEATURE - Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESKC

SEQ ID NO: 115 - protein, 460 aa, synthetic construct
  REGION 1..460: note = Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTKC

SEQ ID NO: 116 - protein, 5 aa, synthetic construct
GYYMH

SEQ ID NO: 117 - protein, 17 aa, synthetic construct
RINPNNGVTLYNQKFKD

SEQ ID NO: 118 - protein, 11 aa, synthetic construct
STMITNYVMDY

SEQ ID NO: 119 - protein, 11 aa, synthetic construct
KASQSVSNDVA

SEQ ID NO: 120 - protein, 7 aa, synthetic construct
YTSSRYA

SEQ ID NO: 121 - protein, 9 aa, synthetic construct
QQDYNSPPT

SEQ ID NO: 122 - protein, 29 aa, synthetic construct
KISGGGGSGGGGSGGGGSGGGGSGGGGSS

SEQ ID NO: 123 - protein, 21 aa, synthetic construct
SPNSASHSGSAPQTSSAPGSQ

SEQ ID NO: 124 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 5 aa, synthetic construct
SFAMS

SEQ ID NO: 126 - protein, 17 aa, synthetic construct
AISGSGGGTYYADSVKG

SEQ ID NO: 127 - protein, 13 aa, synthetic construct
DKILWFGEPVFDY

SEQ ID NO: 128 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 11 aa, synthetic construct
RASQSVSSYLA

SEQ ID NO: 130 - protein, 7 aa, synthetic construct
DASNRAT

SEQ ID NO: 131 - protein, 9 aa, synthetic construct
QQRSNWPPT

SEQ ID NO: 132 - protein, 449 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELIKNQVSLICLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 5 aa, synthetic construct
DYWMQ

SEQ ID NO: 134 - protein, 17 aa, synthetic construct
TIYPGDGDTGYAQKFQG

SEQ ID NO: 135 - protein, 11 aa, synthetic construct
GDYYGSNSLDY

SEQ ID NO: 136 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 11 aa, synthetic construct
KASQDVSTVVA

SEQ ID NO: 138 - protein, 7 aa, synthetic construct
SASYRYI

SEQ ID NO: 139 - protein, 9 aa, synthetic construct
QQHYSPPYT